(19) United States
(12) Patent Application Publication
(10) Pub. No.: US 2006/0134663 A1
(43) Pub. Date: Jun. 22, 2006

(54) Transcriptome microarray technology and methods of using the same

(76) Inventors: Paul Harkin, Antrim (IE); Patrick Johnston, Antrim (IE); Karl Mulligan, Belfast (IE)

Correspondence Address: JOHN S. PRATT, ESQ;KILPATRICK STOCKTON, LLP, 1100 PEACHTREE STREET, ATLANTA, GA, 30309, US

(21) Appl. No.: 11/266,748

(22) Filed: Nov. 3, 2005

Related U.S. Application Data
(60) Provisional application No. 60/662,276, filed on Mar. 14, 2005.
(60) Provisional application No. 60/700,293, filed on Jul. 18, 2005.

(30) Foreign Application Priority Data
Nov. 3, 2004 (EP) 04105479.2
Nov. 3, 2004 (EP) 04105482.6
Nov. 3, 2004 (EP) 04105483.4
Nov. 3, 2004 (EP) 04105484.2
Nov. 3, 2004 (EP) 04105507.0
Nov. 3, 2004 (EP) 04105485.9

(51) Int. Cl. C12Q 1/68; C12M 1/34

(52) U.S. Cl. 435/6; 435/287.2

(57) ABSTRACT

Arrays containing a transcriptome of a diseased tissue and methods of using the arrays for diagnosis, prognosis, screening, and identification of disease are provided herein. The transcriptome arrays from diseased tissue are useful for diagnosis of a disease by analysis of the genetic profile of a tissue sample specific to a disease state. The genetic profiles are then correlated with data on the effectiveness of specific therapeutic agents. Correlating expression profiles to the effectiveness of therapeutic agents provides a way to screen and select further patients predicted to respond to those therapeutic agents, thereby minimizing needless exposure to ineffective therapy.

### CLAIM OF PRIORITY AND CROSS-REFERENCE TO RELATED APPLICATIONS

This application claims priority of European Patent Application No. 04105479.2 filed Nov. 3, 2004, European Patent Application No. 04105482.6 filed Nov. 3, 2004, European Patent Application No. 04105483.4 filed Nov. 3, 2004, European Patent Application No. 04105484.2 filed Nov. 3, 2004, European Patent Application No. 04105507.0 filed Nov. 3, 2004, European Patent Application No. 04105485.9 filed Nov. 3, 2004, and to U.S. Provisional Patent Application No. 60/662,276 filed Mar. 14, 2005, and U.S. Provisional Patent Application No. 60/700,293 filed Jul. 18, 2005, each of which is incorporated herein by reference.

### FIELD OF THE INVENTION

This relates to the field of gene and RNA expression array technology, and more particularly relates to arrays containing transcripts expressed in diseased tissue and their use in diagnosis and therapy decisions.

### REFERENCE TO DOCUMENTS CO-FILED ON CD-R

A total of three (3) identical CD-R discs (labeled “Copy 1”, “Copy 2” and “Copy 3”) are submitted herewith each containing the following electronic text files. The CD-R discs were created on Nov. 2, 2005, and the sizes of each file are listed parenthetically as follows. All electronic files on the CD-R discs are herein incorporated by reference in their entirety.

| | | |
|---|---|---|
| GeneListA.txt | (30.7 | Mb) |
| GeneListB.txt | (1.9 | Mb) |
| GeneListC.txt | (2 | Mb) |
| GeneListD.txt | (1.1 | Mb) |
| GeneListE.txt | (58.6 | Mb) |
| GeneListF.txt | (3.5 | Mb) |
| GeneListG.txt | (30.7 | Mb) |
| GeneListH.txt | (4.1 | Mb) |
| GeneListI.txt | (30 | Mb) |
| GeneListJ.txt | (18 | kb) |
| GeneListK.txt | (20 | kb) |
| GeneListL.txt | (9.7 | Mb) |
| GeneListM.txt | (5.1 | Mb) |
| GeneListN.txt | (238 | kb) |
| GeneListO.txt | (35.8 | Mb) |
| GeneListP.txt | (11.8 | Mb) |
| GeneListQ.txt | (3.9 | Mb) |
| GeneListR.txt | (10.1 | Mb) |
| GeneListS.txt | (6.1 | Mb) |
| GeneListT.txt | (29.6 | Mb) |
| GeneListU.txt | (1.7 | Mb) |
| GeneListV.txt | (13.3 | Mb) |
| GeneListW.txt | (18.9 | Mb) |
| GeneListX.txt | (10 | kb) |
| GeneListY.txt | (28 | kb) |
| GeneListZ.txt | (5.7 | Mb) |
| GeneListAA.txt | (14.6 | Mb) |
| GeneListBB.txt | (5.1 | Mb) |
| GeneListCC.txt | (34 | Mb) |
| GeneListDD.txt | (26.6 | Mb) |
| GeneListEE.txt | (4 | kb) |
| GeneListFF.txt | (324 | kb) |
| GeneListGG.txt | (8.6 | Mb) |
| GeneListHH.txt | (18.8 | Mb) |
| GeneListII.txt | (9.6 | Mb) |
| GeneListJJ.txt | (46.1 | Mb) |

A total of three (3) identical CD-R discs (electronic medium labeled “Copy 1—Sequence Listing Part”, “Copy 2—Sequence Listing Part” and “Copy CFR—Sequence Listing Part”) are submitted herewith each containing a Sequence Listing of all of the sequences described herein. Pursuant to 37 CFR 1.823, the Sequence Listing is being submitted on compact disc (CD-R) in compliance with 37 CFR 1.52(e) and in Computer Readable Form on compact disc in accordance with 37 CFR 1.824.

The Sequence Listing submitted on the above-mentioned CD-R discs is herein incorporated by reference in its entirety.

### BACKGROUND OF THE INVENTION

The pharmaceutical industry continuously pursues new drug treatment options that are more effective, more specific or have fewer adverse side effects than currently administered drugs. Drug therapy alternatives are constantly being developed because genetic variability within the human population results in substantial differences in the effectiveness of many drugs. Therefore, although a wide variety of drug therapy options are currently available, more therapies are always needed in the event that a patient fails to respond.

Traditionally, the treatment paradigm used by physicians has been to prescribe a first-line drug therapy that results in the highest success rate possible for treating a disease. Alternative drug therapies are then prescribed if the first is ineffective. This paradigm is clearly not the best treatment method for certain diseases. For example, in diseases such as cancer, the first treatment is often the most important and offers the best opportunity for successful therapy, so there exists a heightened need to chose an initial drug that will be the most effective against that particular patient's disease.

Identification of the optimal first-line drug has been impossible because no method has been available for predicting which drug treatment would be the most effective for a particular cancer's physiology. Therefore, patients often needlessly undergo ineffective, toxic drug therapy. For example, in colorectal cancer, no method exists for determining which patients will respond to adjuvant chemotherapy after surgery. Only one third of the 40% of patients at risk for relapse after surgery derive benefit from chemotherapy. This means that the administration of adjuvant chemotherapy exposes numerous patients to unnecessary treatment. Cancer treatment and colorectal cancer clinical trials are still being pursued on the basis of the availability of new active compounds rather than the integrated approach of pharmacogenomics that utilizes the genetic makeup of the tumor and the genotype of the patient.

The advent of microarrays and molecular genomics has the potential for a significant impact on the diagnostic capability and prognostic classification of disease, which may aid in the prediction of the response of an individual patient to a defined therapeutic regime. Microarrays provide for the analysis of large amounts of genetic information, thereby providing a genetic fingerprint of an individual. There is much enthusiasm that this technology will ultimately provide the necessary tools for custom-made drug treatment regimes. However, problems have been encountered with the ability to assemble the correct information needed to adequately characterize and predict the response of an individual to a particular drug therapy, and the high expectations of applied pharmacogenomics have been met with some disappointment. (Nebert et al. 2003. Am J Pharmacogenomics; 3(6):361-70).

A major problem with current arrays is that they are typically based on generic information content that has been derived from partial sequencing projects that generate Expressed Sequence Tag (EST) information across a range of different tissue types. Alternatively, the information may be generated from genome-based sequencing projects that utilize algorithms to predict the presence of genes. A significant problem with this approach is that microarray manufacturers must constantly update the information content as more sequence information becomes available. This in turn has led to multiple versions of arrays each with more information content than the previous build. This has created a significant barrier to the routine application of this technology in patient management as researchers are faced with multiple different array platforms with different content making data validation extremely difficult. Even within a specific manufactured array platform it is difficult to cross-validate information between earlier and later versions of arrays, which in turn makes long term study design extremely difficult.

Another problem with currently available microarrays is that different forms of a disease may exist that present different responses to different therapeutic agent treatments. The usefulness of arrays is limited by how representative they are of the particular diseased tissue. The conventional whole genome array is therefore disadvantaged because the extraneous signals provided by genes not related to the disease state provide a high volume of experimental noise, thereby complicating analysis of the diseased transcriptome.

Conventional generic arrays provide limited information across multiple tissue types. However, they do not contain detailed information content regarding the specific transcripts expressed in a given discrete setting. The general approach of the generic microarray industry is to increase the density and content of information as more information becomes available. This has caused confusion in the general adoption of this technology in pharmacogenomics-based studies. The major issue relates to the difficulties in comparing studies across different builds of generic array. That is, it is extremely difficult to correlate data derived from a 20 k sequence array with data derived from a 40 k sequence array. This confusion is caused by problems with annotation and differences in control.

### SUMMARY OF THE INVENTION

Arrays containing biological molecules corresponding to transcriptomes from diseased tissues and methods of using the arrays in assays are provided. Arrays containing nucleic acid molecules corresponding to transcriptomes from diseased tissues and methods of using the arrays in assays are described herein. A diseased tissue transcriptome is a collection of nucleic acid transcripts, for both coding and non-coding nucleic acid sequences, expressed in a particular diseased tissue. Arrays containing other biological molecules corresponding to transcriptomes from diseased tissues are also described herein. Such biological molecules include proteins, polypeptides and antibodies. The arrays provide powerful tools for studying the entire expression profile of diseased tissues and identifying novel transcripts related to disease states.

The microarrays described herein provide a solution to the difficulties encountered in previously available arrays by taking the unique approach of defining the complete transcriptome information content in given disease settings and placing this information content onto an array. The complete information content is derived from multiple diseased tissue samples at varying stages of disease progression thereby encompassing population and disease heterogeneity. This approach ensures that all of the relevant information in a given disease setting is available for interrogation thereby dramatically increasing the potential for developing robust signatures that are diagnostic, prognostic or predictive of response to therapy in that given disease setting. In addition, this approach results in the generation of arrays with complete information content that do not require multiple updates and therefore lends itself to long-term stable study design. Furthermore because this approach represents a complete and stable platform it facilitates cross-validation studies across multiple patient populations in a given disease setting.

Disease specific transcriptome arrays contain complete information content in a given disease setting and therefore represent a stable, long term solution for pharmacogenomic-based study design.

In one aspect of the methods provided herein, the transcriptome arrays are useful for diagnosing a disease by determining the genetic profile of a diseased tissue sample from a patient. The genetic profile is determined by reacting transcripts from a diseased tissue sample, or tissue sample suspected of disease, with the transcriptome array. Hybridization or binding of the transcripts with complementary sequences on the array is then detected. Preferably, the transcriptome array is an array immobilized on a computer chip and hybridization of the nucleic acid molecules from sample to the array is detected using computerized technology. The genetic profile of the diseased tissue sample is then correlated with data on the effectiveness and responsiveness of that profile to specific therapeutic agents. A correlation of the resulting expression profile to the effectiveness of therapeutic agents provides a method for screening and selecting further patients predicted to respond to a particular therapeutic agent, thereby minimizing needless patient exposure to unsuccessful therapies.

Another aspect of the present method includes use of the transcriptomes described herein in methods, such as array assays, for detecting an early stage disease or disorder in an organism that is otherwise undetectable. Such organisms include humans, animals, plants or bacteria.

The arrays and methods of using the array, described herein, provide and utilize transcriptomes to detect, monitor and identify numerous diseases and disorders. All disease may be generally grouped into neoplastic diseases, inflammatory diseases and degenerative diseases. These categories include, but are not limited to diseases such as, cancer, arthritis, asthma, neurodegenerative disease, cardiovascular disease, hypertension, psychiatric disorders, infectious diseases, metabolic diseases or immunological disorders.

In one embodiment, a transcriptome array provides what is believed to be the most complete compilation of the colorectal transcriptome identified to date. Approximately 69,000 transcripts derived from colorectal tissue have been assembled to generate a colorectal, transcriptome-based, high density, oligonucleotide array. Approximately 40,000 of these transcripts are described in U.S. provisional patent application Ser. No. 60/662,276, which is incorporated herein by reference in its entirety. Approximately 23,000 additional transcripts and approximately 5,000 antisense transcripts derived from colorectal tissue are described herein to supplement the colorectal transcriptome sequences described in U.S. provisional patent application Ser. No. 60/662,276.

The transcriptomes provided herein for use in the arrays are believed to be the most complete version of transcriptomes identified to date for lung, breast, colon/rectum, liver, and brain tissue. Transcripts have been assembled herein to generate transcriptome-based, high density, oligonucleotide arrays for diseased tissue from lung, breast, colon/rectum, liver, and brain.

Therefore, the arrays described herein provide a vast amount of information on important changes that may underlie disease progression or resistance to therapy.

Pharmacogenomics has the potential to dramatically reduce the estimated 100,000 deaths and two million hospitalizations that occur each year in the United States as the result of adverse drug response (Lazarou et al. JAMA. Apr. 15, 1998. 279(15):1200-5.) Instead of the standard trial and error method of matching patients with drugs, the arrays and assays described herein enable physicians to analyze the genetic profile of a patient sample and prescribe the best available drug therapy for that patient from the initial diagnosis stage. The arrays described herein not only provide a method for improving the accuracy of prescribing the most effective drug first, but also provide increased safety because the likelihood of adverse drug reactions is reduced.

Accordingly, it is an object of the present invention to provide arrays containing nucleic acid arrays of genes, polynucleotides, nucleotides and fragments from diseased tissues for screening the expression of disease-related genes in a target sample.

It is another object of the present invention to provide methods to identify novel nucleic acid transcripts expressed in a diseased tissue.

It is another object of the present invention to provide methods for screening for genetic variants in a tissue that indicate the presence of an otherwise undetectable disease or disorder.

It is another object of the present invention to provide methods to diagnose a disease based on analysis of the transcriptome from a diseased tissue.

It is another object of the present invention to provide methods for a complete analysis of RNA expression changes affecting all identified genes or transcripts in a specific disease.

It is another object of the present invention to provide methods for characterizing an individual's specific gene/RNA expression profile in a diseased tissue and correlate the RNA expression to a suitable and effective drug treatment regime.

It is another object of the present invention to provide methods for differentiating between different forms of a disease and correlate to an expression profile for a successful therapeutic agent treatment regime.

It is a further object of the present invention to provide methods for correlating an expression profile with a suitable and suitable therapeutic agent treatment regime.

It is another object of the present invention to provide methods for predicting the recurrence of cancer after treatment.

These and other objects, features and advantages of the present invention will become apparent after a review of the following detailed description of the disclosed embodiments and the appended claims.

### BRIEF DESCRIPTION OF THE FIGURES

The file of this patent contains FIG. 1 which is executed in color. Copies of this patent with color drawing will be provided by the Office upon request and payment of the necessary fee.

FIG. 1: Provides a diagram of a transcriptome microarray showing the expression profile for a therapeutic agent-sensitive and a therapeutic agent-resistant tumor.

FIG. 2: Provides a schematic diagram of the BLAST comparison of all publicly available data for colon, prostate and breast tissue.

### DETAILED DESCRIPTION

Transcriptome arrays and methods of use are provided herein. Transcriptome arrays containing nucleic acid molecules from diseased tissue transcripts arranged in an array format are described. The nucleic acid molecules on the array hybridize to complementary nucleic acid transcriptome sequences from a diseased tissue sample. A disease specific transcriptome is defined herein as a collection of coding and non-coding transcripts transcribed in a specific diseased tissue. Additional arrays are described herein that contain other biological molecules, such as polypeptides or antibodies, representative of transcripts from diseased tissue transcriptomes.

Thus, the arrays provided herein encompass nucleic acid arrays, polypeptide arrays, or antibody arrays. In this specification, unless the context demands otherwise, where specific embodiments are described with reference to nucleic acid arrays, it should be understood that corresponding protein arrays and antibody arrays are also contemplated. In such embodiments, the nucleic acids are replaced by polypeptides encoded by the transcripts or antibodies specific for the polypeptides.

The compositions and methods described herein may be understood more readily by reference to the following detailed description of specific embodiments. Although the compositions and methods have been described with reference to specific details of certain embodiments thereof, it is not intended that such details should be regarded as limitations upon the scope of the invention. The text of the references mentioned herein are hereby incorporated by reference in their entirety, including European patent applications EP 04105479.2, EP 04105482.6, EP 04105483.4, EP 04105484.2, EP 04105507.0, and EP 04105485.9 and U.S. provisional patent application 60/662,276 and 60/700,293.

It is well understood by those skilled in the art that cellular DNA in the form of genes is transcribed into RNA; coding RNA is translated into proteins; and RNA is optionally reverse-transcribed into cDNA. Preferably, the transcriptome array described herein contains all or substantially all of the RNA transcripts of the diseased tissue.

The disease specific transcriptome contains transcripts of known and unknown function and optionally includes proteins translated from coding RNA transcripts as an extension and reflection of gene transcription within the transcriptome. The disease specific transcriptome may change as the disease progresses or in response to external stimuli or influence such as chemotherapy or radiotherapy treatment.

As used herein, the term “transcript” means an RNA molecule that is derived through the process of transcription from a DNA or a cDNA template. Transcripts may also be represented by proteins translated from RNA transcripts or cDNA molecules that are reverse-transcribed from RNA transcripts.

As used herein, the term “gene product” means both RNA molecules derived through the process of transcription from a DNA or a cDNA template and polypeptide molecules that are translated from such RNA molecules.

As used herein, the term “transcriptome” means a collection of RNA transcripts transcribed in a specific tissue, whether coding or non-coding, and preferably contains all or substantially all of the RNA transcripts generated in the tissue. These transcripts include messenger RNAs (mRNA), alternatively spliced mRNAs, ribosomal RNA (rRNA), transfer RNAs (tRNAs) in addition to a large range of other transcripts, which are not translated into protein such as small nuclear RNAs (snRNAs), antisense molecules such as short interfering RNA (siRNA) and microRNA and other RNA transcripts of unknown function. The transcriptome also includes proteins translated from the RNA transcripts within the transcriptome, which is an extension and reflection of gene transcription within the transcriptome.

As used herein, the term “diseased tissue” means tissue derived from a particular organ or tissue type which has a particular class of disease associated with the tissue. (e.g. colorectal cancer, breast cancer, neurodegenerative disease, etc.). Diseased tissue may also refer to individual cell types such as epithelial cells, stromal cells or stem cells all derived from that diseased tissue. For example, diseased colorectal tissue is any colorectal tissue that has been diagnosed as having a disease or disorder such as cancer. In most embodiments of the present transcriptome array, no attempt is made to differentiate different types of cancer in the tissue, although in certain embodiments, differentiation of cancer type may be performed.

In addition, it is to be understood that in sampling diseased tissue, there may be a some normal, non-diseased tissue or cells that are sampled with the diseased tissue.

Nucleic Acids

The nucleic acid molecules, nucleic acid elements or polynucleotides composing the arrays provided herein may be any type of nucleic acid or nucleic acid analog, including without limitation, RNA, DNA, peptide nucleic acids, or mixtures and/or fragments thereof. As used herein the term “fragment” refers to a nucleotide sequence that is a part of a sequence such as those provided herein that retains sufficient nucleotide sequence to permit the fragment to maintain specificity and selectivity to the whole sequence from which it is derived. Fragments may be complementary to the whole sequence and retain the ability to selectively hybridize to the whole sequence. The nucleic acid molecules are isolated, cloned or synthetically produced. The nucleic acid elements may include vector sequences or may be substantially pure. The nucleic acid elements are capable of hybridizing, under conventional hybridization conditions, to complementary transcripts in a nucleic acid sample containing transcript-specific molecules or elements derived from a tissue sample. One of ordinary skill in the art may adjust hybridization factors to provide optimum hybridization and signal production for a given hybridization procedure and to provide the required resolution among different genes or genomic locations.

The following lists of transcripts provide sequences specific to a particular diseased tissue. The lists are summarized in Table 1 below. The term “gene list” as used in this table and throughout this specification means “nucleic acid transcript list” and includes both coding and non-coding regions.

TABLE 1

Summary of Sequence Listing Transcript Lists

Number of

| | | |
|---|---|---|
| Tissue/Gene List | Sequences | Sequence Listing Range |

| | | |
|---|---|---|
| Colorectal Sequences | | |
| Gene List A | 16,350 | SEQ ID NO: 1 to SEQ ID NO: 16,350 |
| Gene List B | 2,773 | SEQ ID NO: 16,351 to SEQ ID NO: 19,123 |
| Gene List C | 1,805 | SEQ ID NO: 19,124 to SEQ ID NO: 20,928 |
| Gene List D | 1,318 | SEQ ID NO: 20,929 to SEQ ID NO: 22,246 |
| Gene List E | 10,556 | SEQ ID NO: 22,247 to SEQ ID NO: 32,802 |
| Gene List F | 7,134 | SEQ ID NO: 32,803 to SEQ ID NO: 39,936 |
| Gene List G | 22,376 | SEQ ID NO: 39,937 to SEQ ID NO: 62,312 |
| Gene List H | 5,672 | SEQ ID NO: 62,313 to SEQ ID NO: 67,984 |
| Lung Sequences | | |
| Gene List I | 36,431 | SEQ ID NO: 67,985 to SEQ ID NO: 104,415 |
| Gene List J | 24 | SEQ ID NO: 104,416 to SEQ ID NO: 104,439 |
| Gene List K | 22 | SEQ ID NO: 104,440 to SEQ ID NO: 104,461 |
| Gene List L | 9,727 | SEQ ID NO: 104,462 to SEQ ID NO: 114,188 |
| Gene List M | 5,208 | SEQ ID NO: 114,189 to SEQ ID NO: 119,396 |
| Gene List N | 452 | SEQ ID NO: 119,397 to SEQ ID NO: 119,848 |
| Gene List O | 42,790 | SEQ ID NO: 119,849 to SEQ ID NO: 162,638 |
| Breast Sequences | | |
| Gene List P | 17,291 | SEQ ID NO: 162,639 to SEQ ID NO: 179,929 |
| Gene List Q | 3,278 | SEQ ID NO: 179,930 to SEQ ID NO: 183,207 |
| Gene List R | 6,915 | SEQ ID NO: 183,208 to SEQ ID NO: 190,122 |
| Gene List S | 4,857 | SEQ ID NO: 190,123 to SEQ ID NO: 194,979 |
| Gene List T | 34,141 | SEQ ID NO: 194,980 to SEQ ID NO: 229,120 |
| Gene List U | 3,911 | SEQ ID NO: 229,121 to SEQ ID NO: 233,031 |
| Gene List V | 16,666 | SEQ ID NO: 233,032 to SEQ ID NO: 249,697 |
| Liver Sequences | | |
| Gene List W | 24,744 | SEQ ID NO: 249,698 to SEQ ID NO: 274,441 |
| Gene List X | 13 | SEQ ID NO: 274,442 to SEQ ID NO: 274,454 |
| Gene List Y | 32 | SEQ ID NO: 274,455 to SEQ ID NO: 274,486 |
| Gene List Z | 6,565 | SEQ ID NO: 274,487 to SEQ ID NO: 281,051 |
| Gene List AA | 14,789 | SEQ ID NO: 281,052 to SEQ ID NO: 295,840 |
| Gene List BB | 11,851 | SEQ ID NO: 295,841 to SEQ ID NO: 307,691 |
| Gene List CC | 39,979 | SEQ ID NO: 307,692 to SEQ ID NO: 347,670 |
| Brain Sequences | | |
| Gene List DD | 33,275 | SEQ ID NO: 347,671 to SEQ ID NO: 380,945 |
| Gene List EE | 5 | SEQ ID NO: 380,946 to SEQ ID NO: 380,950 |
| Gene List FF | 341 | SEQ ID NO: 380,951 to SEQ ID NO: 381,291 |
| Gene List GG | 8,486 | SEQ ID NO: 381,292 to SEQ ID NO: 389,777 |
| Gene List HH | 19,081 | SEQ ID NO: 389,778 to SEQ ID NO: 408,858 |
| Gene List II | 21,845 | SEQ ID NO: 408,859 to SEQ ID NO: 430,703 |
| Gene List JJ | 53,293 | SEQ ID NO: 430,704 to SEQ ID NO: 483,996 |

The sequences in each of Gene Lists A-JJ are included with this specification on the enclosed CD-R and are incorporated herein by reference in their entirety.

Transcripts from Diseased Colorectal Tissue

Gene List A (SEQ ID NO:1 to SEQ ID NO:16,350)

A collection of 16,350 transcripts that have been previously identified as being expressed in colorectal tissue are provided herein.

Accordingly, in one embodiment, an array of nucleic acid molecules complementary to at least 4,000 of the nucleic acid molecules set forth in Gene List A is provided. In other embodiments, the array contains nucleic acid molecules complementary to at least 6,000, 8,000, 10,000, 12,000, 14,000 or 16,000 of the sequences set forth in Gene List A.

Gene List B (SEQ ID NO:16,351 to SEQ ID NO:19,123)

An assembly of 2,773 transcripts that do not hit against either publicly available expressed sequence tag (EST) libraries generated from colorectal cancer or annotated genes in Genbank are described. These genes are newly identified herein.

Accordingly, in one embodiment, an array containing nucleic acid molecules complementary to at least 1,000 of the nucleic acid molecules set forth in Gene List B is provided. In other embodiments, the array contains nucleic acid molecules complementary to at least 50, 100, 500, 1,000, 1,500, 2000, or 2500 of the sequences set forth in Gene List B.

Gene List C (SEQ ID NO:19,124 to SEQ ID NO:20,928)

A cDNA library has been generated from diseased human colorectal tissues, and 1,805 nucleotide sequences have been identified herein by high throughput sequencing, which have not previously been identified as being expressed in colorectal cancer tissue.

Accordingly, in one embodiment, an array of nucleic acid molecules complementary to at least 500 of the nucleic acid molecules set forth in Gene List C is provided. In other embodiments, the array contains nucleic acid molecules complementary to at least 50, 200, 500, 750, 1,000, 1,400, or 1,750 of sequences set forth in Gene List C.

Gene List D (SEQ ID NO:20,929 to SEQ ID NO:22,246)

Alternative pre-mRNA splicing is a major cellular process by which functionally diverse proteins can be generated from the primary transcript of a single gene, often in tissue specific patterns.

A collection of 1,318 nucleotide sequences that exist as significantly altered (spliced) forms of previously annotated genes or ESTs, which are expressed in colorectal cancer tissues, have been newly identified herein. Accordingly in one embodiment, an array containing nucleic acid molecules complementary to at least 500 of the nucleic acid molecules set forth in Gene List D is provided. In other embodiments, the array contains nucleic acid molecules complementary to at least 50, 100, 250, 500, 750, 1,000, or 1,250 of the sequences set forth in Gene List D.

Gene List E (SEQ ID NO:22,247 to SEQ ID NO:32,802)

A cDNA library has been generated from diseased human colorectal tissues, and 10,556 nucleotide sequences have been identified herein, which have not previously been identified as being expressed in colorectal cancer tissue.

Accordingly, in one embodiment, an array of nucleic acid molecules complementary to at least 500 of the nucleic acid molecules set forth in Gene List E is provided. In other embodiments, the array contains nucleic acid molecules complementary to at least 1,000, 2,000, 5,000, or 10,000 of sequences set forth in Gene List E.

Gene List F (SEQ ID NO:32,803 to SEQ ID NO:39,936)

A cDNA library has been generated from diseased human colorectal tissues, and 7,134 nucleotide sequences have been identified herein, which have not previously been identified as annotated genes.

Accordingly, in one embodiment, an array of nucleic acid molecules complementary to at least 500 of the nucleic acid molecules set forth in Gene List F is provided. In other embodiments, the array contains nucleic acid molecules complementary to at least 1,000, 2,500, 5,000, or 7,000 of sequences set forth in Gene List F.

Gene List G (SEQ ID NO:39,937 to SEQ ID NO:62,312)

A collection of 22,376 nucleotide sequences have been identified herein, which have not previously been identified as being expressed in colorectal cancer tissue.

Accordingly, in one embodiment, an array of nucleic acid molecules complementary to at least 4,000 of the nucleic acid molecules set forth in Gene List G is provided. In other embodiments, the array contains nucleic acid molecules complementary to at least 6,000, 8,000, 10,000, 12,000, 14,000, 16,000, or 19,000 of sequences set forth in Gene List G.

Gene List H (SEQ ID NO:62,313 to SEQ ID NO:67,984)

A collection of 5,672 nucleotide sequences have been newly identified herein that constitute antisense and corresponding reverse complement transcripts.

The inclusion of antisense transcripts and their corresponding sense transcripts is an important feature of the array. Generic commercially available arrays focus primarily on measuring sense protein coding transcripts. With the increasing interest in the role of endogenous antisense RNA transcripts in cancer and other diseases, antisense sequences within the colorectal transcriptome have now been identified.

Accordingly, in one embodiment, an array of nucleic acid molecules complementary to at least 2,000 of the nucleic acid molecules set forth in Gene List H is provided. In other embodiments, the array contains nucleic acid molecules complementary to at least 3,000, 4,000, or 5,000 of the sequences set forth in Gene List H.

Transcripts from Diseased Lung Tissue

Gene List I (SEQ ID NO:67,985 to SEQ ID NO:104,415)

A collection of 36,431 transcripts previously shown to have been implicated in lung cancer are provided herein.

Accordingly, in one embodiment, an array of nucleic acid molecules complementary to at least 4,000 of the nucleic acid molecules set forth in Gene List I is provided. In other embodiments, the array contains nucleic acid molecules complementary to at least 6,000, 8,000, 15,000, 20,000, 30,000, or 35,000 of the sequences set forth in Gene List I.

Gene List J (SEQ ID NO:104,416 to SEQ ID NO:104,439)

An assembly of 24 transcripts that do not hit against either publicly available EST libraries generated from lung cancer tissue or annotated genes in Genbank are described. These genes are newly identified herein.

Accordingly, in one embodiment, an array of nucleic acid molecules complementary to at least 5 of the nucleic acid molecules set forth in Gene List J is provided. In other embodiments, the array contains nucleic acid molecules complementary to at least 6, 10, 15, 18, 20 or 22 of the sequences set forth in Gene List J.

Gene List K (SEQ ID NO:104,440 to SEQ ID NO:104,461)

A collection of 22 expressed sequence tags identified by high throughput sequencing that have not previously been reported to be expressed in lung tissue have been identified herein.

Accordingly, in one embodiment, an array of nucleic acid molecules complementary to at least 5 of the nucleic acid molecules set forth in Gene List K is provided. In other embodiments, the array contains nucleic acid molecules complementary to at least 6, 10, 15, 18, or 20 of the sequences set forth in Gene List K.

Gene List L (SEQ ID NO:104,462 to SEQ ID NO:114,188)

A collection of 9,727 transcripts identified as containing sequences that exist as a significantly altered (spliced) form of previously annotated lung cancer-associated genes or ESTs have been newly identified herein.

Accordingly, in one embodiment, an array of nucleic acid molecules complementary to at least 3,000 of the nucleic acid molecules set forth in Gene List L is provided. In other embodiments, the array contains nucleic acid molecules complementary to at least 4,000, 5,000, 7,000, or 9,000 of the sequences set forth in Gene List L.

Gene List M (SEQ ID NO:114,189 to SEQ ID NO:119,396)

A collection of 5,208 annotated genes that have been identified as being expressed in diseased lung tissue has been newly identified herein.

Accordingly, in one embodiment, an array of nucleic acid molecules complementary to at least 2,500 of the nucleic acid molecules set forth in Gene List M is provided. In other embodiments, the array contains nucleic acid molecules complementary to at least 3,000, 4,000, or 5,000 of the sequences set forth in Gene List M.

Gene List N (SEQ ID NO:119,397 to SEQ ID NO:119,848)

A collection of 452 transcripts were identified herein as singlet nucleotide sequences, which are expressed in lung cancer tissue and which have not previously been identified as annotated genes.

Accordingly, in one embodiment, an array of nucleic acid molecules complementary to at least 200 of the nucleic acid molecules set forth in Gene List N is provided. In other embodiments, the array contains nucleic acid molecules complementary to at least 250, 300, 350 or 400 of the sequences set forth in Gene List N.

Gene List O (SEQ ID NO:119,849 to SEQ ID NO:162,638)

A collection of 42,790 transcripts that constitute antisense and corresponding reverse complement transcripts for sequences expressed in lung cancer tissue have been newly identified herein.

Accordingly, in one embodiment, an array of nucleic acid molecules complementary to at least 20,000 of the nucleic acid molecules set forth in Gene List O is provided. In other embodiments, the array contains nucleic acid molecules complementary to at least 25,000, 30,000, 35,000, or 40,000 of the sequences set forth in Gene List O.

Transcripts from Diseased Breast Tissue

Gene List P (SEQ ID NO:162,639 to SEQ ID NO:179,929)

A collection of 17,291 expressed sequence tags that have been previously shown to be expressed in breast cancer tissue are provided herein.

Accordingly, in one embodiment, an array of nucleic acid molecules complementary to at least 3,000 of the nucleic acid molecules set forth in Gene List P is provided. In other embodiments, the array contains nucleic acid molecules complementary to at least 4,000, 5,000, 7,000, 10,000, 12,000, 15,000, or 17,000 of the sequences set forth in Gene List P.

Gene List Q (SEQ ID NO:179,930 to SEQ ID NO:183,207)

An assembly of 3,278 transcripts that do not hit against either publicly available EST libraries generated from breast cancer tissue or annotated genes in Genbank are described. These genes are newly identified herein.

Accordingly, in one embodiment, an array of nucleic acid molecules complementary to at least 1,000 of the nucleic acid molecules set forth in Gene List Q is provided. In other embodiments, the array contains nucleic acid molecules complementary to at least 2,000 or 3,000 of the sequences set forth in Gene List Q.

Gene List R (SEQ ID NO:183,208 to SEQ ID NO:190,122)

An assembly of 6,915 transcripts identified by high throughput sequencing that have not previously been reported to be expressed in diseased breast tissue have been identified herein.

Accordingly, in one embodiment, an array of nucleic acid molecules complementary to at least 2,000 of the nucleic acid molecules set forth in Gene List R is provided. In other embodiments, the array contains nucleic acid molecules complementary to at least 4,000 or 6,000 of the sequences set forth in Gene List R.

Gene List S (SEQ ID NO:190,123 to SEQ ID NO:194,979)

An assembly of 4,857 transcripts identified as containing sequences that exist in diseased breast tissue as a significantly altered (spliced) form of previously annotated genes or ESTs have been newly identified herein.

Accordingly, in one embodiment, an array of nucleic acid molecules complementary to at least 1,000 of the nucleic acid molecules set forth in Gene List S is provided. In other embodiments, the array contains nucleic acid molecules complementary to at least 2,000 or 4,000 of the sequences set forth in Gene List S.

Gene List T (SEQ ID NO:194,980 to SEQ ID NO:229,120)

An assembly of 34,141 transcripts have been identified herein as being expressed in breast tissue. These transcripts were not previously confirmed as being expressed in breast cancer tissue.

Accordingly, in one embodiment, an array of nucleic acid molecules complementary to at least 10,000 of the nucleic acid molecules set forth in Gene List T is provided. In other embodiments, the array contains nucleic acid molecules complementary to at least 15,000, 20,000, 25,000, or 30,000 of the sequences set forth in Gene List T.

Gene List U (SEQ ID NO:229,121 to SEQ ID NO:233,031)

An assembly of 3,911 transcripts were identified herein as singlet nucleotide sequences, which are expressed in breast cancer tissue and which have not previously been identified as annotated genes.

Accordingly, in one embodiment, an array of nucleic acid molecules complementary to at least 1,000 of the nucleic acid molecules set forth in Gene List U is provided. In other embodiments, the array contains nucleic acid molecules complementary to at least 1,500, 2,000, 2,500 or 3,000 of the sequences set forth in Gene List U.

Gene List V (SEQ ID NO:233,032 to SEQ ID NO:249,697)

An assembly of 16,666 transcripts that constitute antisense and corresponding sense transcripts for sequences expressed in breast cancer tissue has been newly identified herein.

Accordingly, in one embodiment, an array of nucleic acid molecules complementary to at least 8,000 of the nucleic acid molecules set forth in Gene List V is provided. In other embodiments, the array contains nucleic acid molecules complementary to at least 10,000, 12,000, 14,000, or 16,000 of the sequences set forth in Gene List V.

Transcripts from Diseased Liver Tissue

Gene List W (SEQ ID NO:249,698 to SEQ ID NO:274,441)

An assembly of 24,744 transcripts that have previously been identified as being expressed in liver tissue associated with hepatitis are provided herein.

Accordingly, in one embodiment, an array of nucleic acid molecules complementary to at least 4,000 of the nucleic acid molecules set forth in Gene List W is provided. In other embodiments, the array contains nucleic acid molecules complementary to at least 6,000, 8,000, 10,000, 12,000, 14,000, 16,000, 19,000, or 21,000 of the sequences set forth in Gene List W.

Gene List X (SEQ ID NO:274,442 to SEQ ID NO:274,454)

An assembly of 13 transcripts that do not hit against either publicly available EST libraries generated from liver tissue associated with hepatitis or annotated genes in Genbank are described herein. These genes are newly identified herein.

Accordingly, in one embodiment, an array of nucleic acid molecules complementary to at least 8 of the nucleic acid molecules set forth in Gene List X is provided. In other embodiments, the array contains nucleic acid molecules complementary to at least 10 or 12 of the sequences set forth in Gene List X.

Gene List Y (SEQ ID NO:274,455 to SEQ ID NO:274,486)

An assembly of 32 transcripts previously identified by high throughput screening but not previously reported to be expressed in liver tissue associated with hepatitis have been identified herein.

Accordingly, in one embodiment, an array of nucleic acid molecules complementary to at least 15 of the nucleic acid molecules set forth in Gene List Y is provided. In other embodiments, the array contains nucleic acid molecules complementary to at least 20, 25, or 30 of the sequences set forth Gene List Y.

Gene List Z (SEQ ID NO:274,487 to SEQ ID NO:281,051)

An assembly of 6,565 transcripts that exist as significantly altered (spliced) forms of previously annotated genes or ESTs and are expressed in liver tissue associated with hepatitis have been identified herein.

Accordingly, in one embodiment, an array of nucleic acid molecules complementary to at least 3,000 of the nucleic acid molecules set forth in Gene List Z is provided. In other embodiments, the array contains nucleic acid molecules complementary to at least 4,000, 5,000, or 6,000 of the sequences set forth in Gene List Z.

Gene List AA (SEQ ID NO:281,052 to SEQ ID NO:295,840)

An assembly of 14,789 transcripts have been newly identified herein as being expressed in liver tissue associated with hepatitis.

Accordingly, in one embodiment, an array of nucleic acid molecules complementary to at least 8,000 of the nucleic acid molecules set forth in Gene List AA is provided. In other embodiments, the array contains nucleic acid molecules complementary to at least 8,000, 10,000, 12,000, or 14,000 of the sequences set forth in Gene List AA.

Gene List BB (SEQ ID NO:295,841 to SEQ ID NO:307,691)

An assembly of 11,851 transcripts have been identified herein as singlet nucleotide sequences, which are expressed in liver tissue associated with hepatitis and which have not previously been identified as annotated genes.

Accordingly, in one embodiment, an array of nucleic acid molecules complementary to at least 6,000 of the nucleic acid molecules set forth in Gene List BB is provided. In other embodiments, the array contains nucleic acid molecules complementary to at least 8,000 or 10,000 of the sequences set forth in Gene List BB.

Gene List CC (SEQ ID NO:307,692 to SEQ ID NO:347,670)

An assembly of 39,979 transcripts that constitute antisense and corresponding sense transcripts for sequences expressed in liver tissue associated with hepatitis have been newly identified herein.

Accordingly, in one embodiment, an array of nucleic acid molecules complementary to at least 20,000 of the nucleic acid molecules set forth in Gene List CC is provided. In other embodiments, the array contains nucleic acid molecules complementary to at least 25,000, 30,000 or 35,000 of the sequences set forth in Gene List CC.

Transcripts from Diseased Brain Tissue

Gene List DD (SEQ ID NO:347,671 to SEQ ID NO:380,945)

An assembly of 33,275 transcripts that have been previously identified as being expressed in brain tissue associated with neurodegenerative disease are provided herein.

Accordingly, in one embodiment, an array of nucleic acid molecules complementary to at least 15,000 of the nucleic acid molecules set forth in Gene List DD is provided. In other embodiments, the array contains nucleic acid molecules complementary to at least 20,000, 25,000 or 30,000 of the sequences set forth in Gene List DD.

Gene List EE (SEQ ID NO:380,946 to SEQ ID NO:380,950)

An assembly of five transcripts has been newly identified herein as containing sequences that do not hit against either publicly available EST libraries generated from brain tissue associated with neurodegenerative disease or annotated genes in Genbank. These genes are newly identified herein.

Accordingly, in one embodiment, an array of nucleic acid molecules complementary to at least three of the nucleic acid molecules set forth in Gene List EE is provided.

Gene List FF (SEQ ID NO:380,951 to SEQ ID NO:381,291)

An assembly of 341 transcripts have been identified herein by high throughput sequencing. These transcripts have not previously been reported to be expressed in brain tissue associated with neurodegenerative disease.

Accordingly, in one embodiment, an array of nucleic acid molecules complementary to at least 150 of the nucleic acid molecules set forth in Gene List FF is provided. In other embodiments, the array contains nucleic acid molecules complementary to at least 200 or 300 of the sequences set forth in Gene List FF.

Gene List GG (SEQ ID NO:381,292 to SEQ ID NO:389,777)

An assembly of 8,486 transcripts have been newly identified herein that exist as significantly altered (spliced) forms of previously annotated genes or ESTs and are expressed in brain tissue associated with neurodegenerative disease.

Accordingly, in one embodiment, an array of nucleic acid molecules complementary to at least 4,000 of the nucleic acid molecules set forth in Gene List GG is provided. In other embodiments, the array contains nucleic acid molecules complementary to at least 6,000 or 8,000 of the sequences set forth in Gene List GG.

Gene List HH (SEQ ID NO:389,778 to SEQ ID NO:408,858)

An assembly of 19,081 transcripts that have been newly identified herein as being expressed in brain tissue associated with neurodegenerative disease are provided.

Accordingly, in one embodiment, an array of nucleic acid molecules complementary to at least 8,000 of the nucleic acid molecules set forth in Gene List HH is provided. In other embodiments, the array contains nucleic acid molecules complementary to at least 12,000, 15,000, 17,000, or 19,000 of the sequences set forth in Gene List HH.

Gene List II (SEQ ID NO:408,859 to SEQ ID NO:430,703)

An assembly of 21,845 transcripts have been identified herein as singlet nucleotide sequences, which are expressed in brain tissue associated with neurodegenerative disease and which have not previously been identified as annotated genes.

Accordingly, in one embodiment, an array of nucleic acid molecules complementary to at least 10,000 of the nucleic acid molecules set forth in Gene List II is provided. In other embodiments, the array contains nucleic acid molecules complementary to at least 12,000, 15,000, 17,000, or 20,000 of the sequences set forth in Gene List II.

Gene List JJ (SEQ ID NO:430,704 to SEQ ID NO:483,996)

An assembly of 53,293 transcripts that constitute antisense and corresponding sense transcripts for sequences expressed in brain tissue associated with neurodegenerative disease have been newly identified herein.

Accordingly, in one embodiment, an array of nucleic acid molecules complementary to at least 30,000 of the nucleic acid molecules set forth in Gene List JJ is provided. In other embodiments, the array contains nucleic acid molecules complementary to at least 35,000, 40,000, 45,000 or 50,000 of the sequences set forth in Gene List JJ.

Arrays

As described above, the lists of transcripts provided herein may be used to prepare diseased tissue transcriptome arrays using nucleic acid molecules complementary to the sequences provided herein. The terms “array” and “microarray” are used interchangeably herein. The latter term is frequently used by those skilled in the art to refer to a type of miniature array associated with a computer chip. As used herein, the term “tissue specific element” refers to a biological molecule on the array that binds to a transcript-specific element from a diseased target sample and includes nucleic acid, polypeptide and antibody molecules.

Gene Lists A-H provide the sequences of transcripts associated with diseased colorectal tissue. In one embodiment, an array containing at least one nucleic acid molecule complementary to diseased colorectal tissue transcripts provided in Gene List B, Gene List C, Gene List D, Gene List E, Gene List F, Gene List G, Gene List H, or combinations thereof, is provided. In another embodiment, arrays containing nucleic acid molecules complementary to at least 70%, for example at least 80% or at least 90% of the diseased colorectal tissue transcripts provided in Gene List B, Gene List C, Gene List D, Gene List E, Gene List F, Gene List G, Gene List H, or combinations thereof, are provided. In another embodiment, arrays containing nucleic acid molecules complementary to at least 70%, for example at least 80% or at least 90% of the diseased colorectal tissue transcripts provided in each of Gene List B, Gene List C, Gene List D, Gene List E, Gene List F, Gene List G, and Gene List H, are provided.

Gene Lists I-O provide the sequences of transcripts associated with diseased lung tissue. In one embodiment, an array containing nucleic acid molecules complementary to diseased lung tissue transcripts provided in Gene List J, Gene List K, Gene List L, Gene List M, Gene List N, Gene List O, or combinations thereof, are provided. In another embodiment, an array containing diseased lung tissue nucleic acid molecules complementary to at least 70%, for example at least 80% or at least 90% of the transcripts provided in Gene List J, Gene List K, Gene List L, Gene List M, Gene List N, Gene List O, or combinations thereof, are provided. In another embodiment, an array containing diseased lung tissue nucleic acid molecules complementary to at least 70%, for example at least 80% or at least 90% of the transcripts provided in Gene List J, Gene List K, Gene List L, Gene List M, Gene List N, Gene List O, are provided.

Gene Lists P-V provide the sequences of transcripts associated with diseased breast tissue. In one embodiment, arrays containing nucleic acid molecules complementary to diseased breast tissue transcripts provided in Gene List Q, Gene List R, Gene List S, Gene List T, Gene List U, Gene List V, or combinations thereof, are provided. In other embodiments, arrays comprising nucleic acid molecules complementary to at least 70%, for example at least 80% or at least 90% of the diseased breast tissue transcripts provided in Gene List Q, Gene List R, Gene List S, Gene List T, Gene List U, Gene List V, or combinations thereof, are provided. In another embodiment, arrays comprising nucleic acid molecules complementary to at least 70%, for example at least 80% or at least 90% of the diseased breast tissue transcripts provided in Gene List Q, Gene List R, Gene List S, Gene List T, Gene List U, Gene List V, are provided.

Gene Lists W-CC provide the sequences of transcripts associated with diseased liver tissue. In one embodiment, arrays containing nucleic acid molecules complementary to diseased liver tissue transcripts provided in Gene List X, Gene List Y, Gene List Z, Gene List AA, Gene List BB, Gene List CC, or combinations thereof are provided. In other embodiments, arrays containing nucleic acid molecules complementary to at least 70%, for example at least 80% or at least 90% of the diseased liver tissue transcripts provided in Gene List X, Gene List Y, Gene List Z, Gene List AA, Gene List BB, Gene List CC, or combinations thereof are provided. In another embodiment, arrays containing nucleic acid molecules complementary to at least 70%, for example at least 80% or at least 90% of the diseased liver tissue transcripts provided in Gene List X, Gene List Y, Gene List Z, Gene List AA, Gene List BB, Gene List CC, are provided.

Gene Lists DD-JJ provide the sequences of transcripts associated with diseased brain tissue. In one embodiment, arrays containing nucleic acid molecules complementary to diseased brain tissue transcripts provided in Gene List EE, Gene List FF, Gene List GG, Gene List HH, Gene List II, Gene List JJ, or combinations thereof, are provided. In other embodiments, arrays containing nucleic acid molecules complementary to at least 70%, for example at least 80% or at least 90% of the diseased brain tissue transcripts provided in Gene List EE, Gene List FF, Gene List GG, Gene List HH, Gene List II, Gene List JJ, or combinations thereof, are provided. In another embodiment, arrays containing nucleic acid molecules complementary to at least 70%, for example at least 80% or at least 90% of the diseased brain tissue transcripts provided in Gene List EE, Gene List FF, Gene List GG, Gene List HH, Gene List II, Gene List JJ, are provided.

In another embodiment, an array containing nucleic acid molecules complementary to the nucleic acid sequences provided in Gene List A-H, J-O, and Q-V from two or more different cancer tissues provides an array directed to multiple types of cancer. In other embodiments, arrays comprising nucleic acid molecules complementary to at least 70%, for example at least 80% or at least 90% of the transcripts provided in Gene List A-H, J-O, and Q-V and combinations thereof are also provided.

Preferably, the array in each of the embodiments described herein contains one or more of the nucleic acid molecules newly identified herein or combinations of the nucleic acid molecules newly identified herein. Combinations containing newly identified nucleic acid molecules for a particular disease, type of disease, or broad range of diseases are included.

Expression

To obtain expression of nucleic acid sequences encoding proteins, the sequences are incorporated in a vector having one or more control sequences operably linked to the nucleic acid to control its expression. The vectors optionally include other sequences such as promoters or enhancers to drive the expression of the inserted nucleic acid, nucleic acid sequences so that the peptide is produced as a fusion and/or nucleic acid encoding secretion signals so that the polypeptide produced in the host cell is secreted from the cell.

In another aspect of the invention, there is provided a vector containing an isolated polynucleotide of an aspect of the invention.

In yet another aspect of the invention, there is provided a host cell containing a vector of an aspect of the invention.

Peptides are obtained by transforming the vectors incorporating specific nucleic acid sequences into host cells in which the vector is functional, culturing the host cells so that the peptide is produced, and recovering the peptide from the host cells or the surrounding medium.

Thus, a method of making a polypeptide is included within the scope of the present invention. The method includes expression of the polypeptide from a nucleic acid molecule encoding the polypeptide. This may conveniently be achieved by growing a host cell in a culture medium containing such a vector under appropriate conditions that cause or allow expression of the polypeptide.

Vectors and Host Cells

Suitable vectors can be chosen or constructed that contain appropriate regulatory sequences, including, but not limited to, promoter sequences, terminator fragments, polyadenylation sequences, enhancer sequences, marker genes and other sequences as appropriate.

Vectors may be plasmids, viral e.g. phage, or phagemid, as appropriate. For further details see, for example, MOLECULAR CLONING: A LABORATORY MANUAL: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in CURRENT PROTOCOLS IN MOLECULAR BIOLOGY, Ausubel et al. eds., John Wiley & Sons, 1992.

Systems for the cloning and expression of a polypeptide in a variety of different host cells are well known. Suitable host cells include bacteria, eukaryotic cells such as mammalian and yeast, and baculovirus systems.

Thus, a further aspect of the present invention provides a host cell containing heterologous nucleic acid as disclosed herein.

Array Manufacture

Polynucleotides are used in the design and construction of the transcriptome arrays described herein. In one embodiment, nucleic acid elements are arranged to produce a single transcriptome array, although the array may contain nucleic acid elements corresponding to a plurality of transcriptomes if desired. The transcriptomes may include a plurality of diseased tissue transcripts from one disease or a plurality of diseases. Disease-specific arrays contain transcripts that are transcribed in one given disease setting.

For example, in colorectal cancer, these transcripts may be transcribed in a range of cell types found in the microenvironment of colorectal tumor cells and may include, for example, stromal cells, epithelial cells, lymphocytes, endothelial cells, stem cells, etc. In another embodiment, pre-malignant or malignant cells alter the expression of transcripts within surrounding cells (such as stromal, endothelial or lymphoid cells found in the microenvironment of the tumor) through physical interaction or secretion of specific proteins, and thereby produce transcripts characteristic of colorectal cancer, which are contained on the disease specific array. Furthermore, when utilizing disease specific arrays as tools to identify genetic signatures that are diagnostic, prognostic or predictive, the actual signature may include transcripts that are derived from some or all of these individual cell populations.

The arrays provided herein may be used for any suitable purpose, such as, but not limited to, diagnosis, prognosis, drug therapy, drug screening, and the like. For a given array, each nucleic acid element may be a whole sequence or a sequence fragmented into different lengths. It is not necessary that all fragments constituting a whole sequence be present on the array.

In one embodiment, tissue-specific nucleic acid elements representative of the transcripts and transcript fragments are immobilized on an array at a plurality of physically distinct locations using nucleic acid immobilization or binding techniques well known in the art. The fragments at several physically distinct locations may together compose an entire transcript or discreet portions of the transcript. The fragments may be complementary to contiguous portions of a transcript or discontiguous portions of a transcript. Hybridization of a nucleic acid molecule from a target sample to the fragments on the array is indicative of the presence of the target transcript in the sample. Hybridization and detection of hybridization are performed by routine detection methods well known to those skilled in the art and described in more detail below.

In one embodiment, multiple probes are used that distinguish a target sequence from other nucleic acid sequences in the diseased tissue sample. In some embodiments, at least 2% of a target sequence is represented by the combination of probes on an array. In further embodiments, at least 5%, at least 10%, at least 20%, at least 30%, at least 40%, at least 50%, at least 60%, at least 70%, at least 80%, or at least 90% of a target sequence is represented on an array. Alternatively, at least 60% of a sequence from Gene List A to Gene List JJ is represented by the combination of probes on an array where the sequence is indicative of a larger target sequence or transcript. In further embodiments, at least 70%, at least 80%, or at least 90%, of a transcript from Gene Lists A to Gene List JJ is represented by the combination of probes on an array. Hybridization of nucleic acid fragments in the sample to those on the array is representative of the presence of the full transcript in the tissue sample.

In another embodiment, a nucleic acid element corresponding to a whole transcript or fragment of a whole transcript is immobilized on an array at only one physically distinct location in a “spotted array” format. Multiple copies of the specific nucleic acid element may be bound to the array substrate at the discreet location. Preferably, this type of “spotted array” includes one or more of the nucleic acid molecules newly identified herein.

As mentioned above, the array preferably contains one or more nucleic acid elements corresponding to the transcript-specific elements provided in Gene List A-JJ or fragments thereof. As mentioned above, arrays specific for certain diseases, such as a specific cancer, can be designed to contain all or a predetermined percentage of the transcriptome for that particular disease. For example, in one embodiment, the array may include all or a select subset of the nucleic acid sequences set forth in the Gene Lists provided above associated with a particular disease, such as colon cancer (Gene Lists A-H). In another embodiment, the array can include transcriptomes such as all or a select subset of the nucleic acid sequences set forth in the Gene Lists provided above associated with a general type of disease such as cancer (Gene Lists A-V). In yet another embodiment, the array can include transcriptomes such as all or a select subset of the nucleic acid sequences set forth in the Gene Lists provided above associated with a particular type of organ and disease such as liver tissue associated with hepatitis (Gene Lists W-CC) or brain tissue associated with neurodegenerative disease (Gene Lists DD-JJ).

In another embodiment, the array includes nucleic acid elements corresponding to at least 50% of a given transcript-specific element provided in Gene Lists A-JJ. In other embodiments, the array includes nucleic acid elements corresponding to at least 60%, for example at least 70%, at least 80% or greater than 90% of a given transcript-specific element provided in Gene List A-JJ. Hybridization of a target transcript-specific element from a diseased tissue sample to the corresponding nucleic acid element on the array is indicative of the presence of the target gene in the sample. Other nucleic acid elements or fragments thereof that correspond to other transcripts provided in Gene Lists A-JJ may be localized to other individual physically distinct locations on the array.

One of skill in the art will appreciate that nucleic acid elements on a given array are complementary to the transcript-specific sequences in a given target sample. Arrays containing the native sequences may also be designed to identify the presence of antisense molecules in a target sample. Endogenous antisense RNA transcripts are of interest because recent literature has implicated endogenous antisense in cancer and other diseases.

In one embodiment, the array is an array of nucleic acid elements representative of a diseased colorectal tissue transcriptome, diseased lung tissue transcriptome or diseased breast tissue transcriptome. In such an array it is preferred that more than 75%, 80%, 90%, 95%, or 98% of the total number of transcripts transcribed in a diseased colorectal, lung or breast tissue transcriptome, are present respectively. In some embodiments, the remaining nucleic acid elements are control elements.

The arrays provided herein for use in the assays described herein are constructed using suitable techniques known in the art. See, for example, U.S. Pat. Nos. 5,486,452; 5,830,645; 5,807,552; 5,800,992 and 5,445,934. In each array, individual nucleic acid elements may appear only once or may be replicated. The arrays may optionally also include control nucleic acid elements.

Any suitable substrate can be used as the solid phase to which the nucleic acid elements are immobilized or bound. For example, the substrate can be glass, plastics, metal, a metal-coated substrate or a filter of any material. The substrate surface may be of any suitable configuration. For example the surface may be planar or may have ridges or grooves to separate the nucleic acid elements immobilized on the substrate. In an alternative embodiment, the nucleic acids are attached to beads, which are separately identifiable. The nucleic acid elements are attached to the substrate in any suitable manner that makes them available for hybridization, including covalent or non-covalent binding.

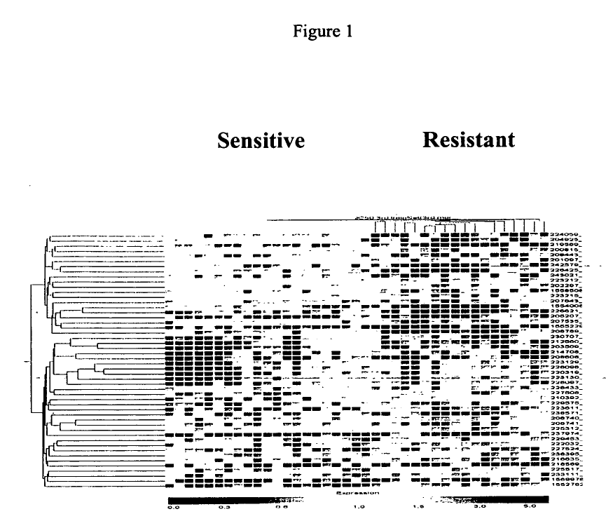

In another embodiment, the polynucleotide or protein molecules in the transcriptome may be grouped on the array by whether the expression of a transcript correlates with sensitivity or resistance to a particular therapeutic agent. Such groupings provide regions on the array where a collection of transcripts is indicative of whether an individual with a particular array profile will be responsive or unresponsive to a particular therapeutic agent (for example see FIG. 1).

Diseased Tissue Sample

Any suitable target tissue or cell may be used as the diseased tissue sample in the methods described herein. It will be understood by those skilled in the art that the term “diseased tissue sample” includes abnormal samples, samples suspected of being diseased, and normal samples that are analyzed as part of a routine screening examination.

The diseased tissue sample is preferably processed to obtain one or more transcript-specific elements, which are then combined with the array to allow hybridization and detection of transcript-specific elements bound to the array. The term “transcript-specific element” as used herein, includes any suitable nucleic acid derived from an RNA transcript in the sample, such as DNA or RNA. The nucleic acid derived from the RNA transcript may be a cDNA reverse-transcribed from an mRNA, an RNA transcribed from such cDNA, a DNA amplified from the cDNA, an RNA transcribed from the amplified DNA, etc. Where it is of interest to determine alterations in the copy number of a gene, genomic DNA is preferably utilized. Alternatively, where expression levels of a transcript or transcripts are to be detected, RNA or cDNA is preferably used. For example, in order to quantify expression, the transcript-specific element may be any type of transcribed RNA molecule such as messenger RNA (mRNA), alternatively spliced mRNA, ribosomal RNA (rRNA), transfer RNA (tRNA), and a large range of other transcripts which are not translated into protein, such as small nuclear RNA (snRNA), and antisense molecules such as siRNA and microRNA. The transcript-specific element may also be a nucleic acid derived from RNA.

A person of ordinary skill in the art will select the appropriate diseased target cell or tissue depending on the purpose of the method. For example, in methods to identify transcripts associated with a particular pathological condition, any biological sample or cell or tissue known to display or express symptoms of the pathological condition may be used.

The arrays described herein are useful for identifying transcripts that are differentially induced in cancers. In such cases the target cells may be tumor cells, for example colon cancer cells or stomach cancer cells. The target cells are derived from any tissue source, including human and animal tissue, such as, but not limited to, a newly obtained sample, a frozen sample, a biopsy sample, a sample of bodily fluid, a blood sample, preserved tissue such as a paraffin-embedded fixed tissue sample (i.e., a tissue block), or cell culture.

For diagnosis, the diseased tissue test sample is preferably derived from a biological sample obtained from an individual suspected of being afflicted with a disease. The tissue sample ideally corresponds to and is combined with the array that contains a substantial portion of one or more complete transcriptomes from the same tissue. The term “substantial portion” is defined herein as approximately greater than 50%, 75%, 80%, 90%, 95%, or 98% of a complete transcriptome. For example, for a diagnosis of lung cancer, transcript-specific elements from a lung tissue sample is applied to an array containing all or a substantial portion of a complete transcriptome for diseased lung tissue.

The population of transcript-specific elements may be obtained from the diseased target tissues or cells using any suitable nucleic acid separation or purification process known in the art. For example, commercially available kits for nucleic acid separation, such as the QIAAMP® tissue kit for DNA isolation from QIAGEN®, (Alameda, Calif.) are useful in the methods described herein. In addition, methods of isolation and purification of nucleic acids are described in Chapter 3 of LABORATORY TECHNIQUES IN BIOCHEMISTRY AND MOLECULAR BIOLOGY: HYBRIDIZATION WITH NUCLEIC ACID PROBES, PART I. THEORY AND NUCLEIC ACID PREPARATION, P. Tijssen, ed. Elsevier, N.Y. (1993).

Depending on the sample size and method of isolation, the transcript-specific elements obtained may be used with or without amplification. Suitable amplification methods include, but are not limited to, polymerase chain reaction (PCR) (Innis, et al., PCR PROTOCOLS: A GUIDE TO METHODS AND APPLICATION, Academic Press, Inc. San Diego, (1990)), ligase chain reaction (LCR) (see Wu and Wallace, Genomics, 4:560 (1989), Landegren, et al., Science, 241:1077 (1988) and Barringer, et al., Gene, 89:117 (1990)), transcription amplification (Kwoh, et al., Proc. Natl. Acad. Sci. USA, 86:1173 (1989)), and self-sustained sequence replication (Guatelli, et al., Proc. Nat. Acad. Sci. USA, 87:1874 (1990)). Details relating to quantitative PCR are provided in PCR PROTOCOLS: A GUIDE TO METHODS AND APPLICATIONS, Innis et al., Academic Press, Inc. N.Y., (1990).

In certain embodiments, only the presence or absence of a particular transcript-specific element need be detected. In such cases, the detection of a hybridization signal is indicative of the presence of the transcript-specific elements in the sample. In other embodiments, it may be desired to quantify the expression of one or more transcript-specific elements in a sample. In such cases, the concentration of transcript-specific elements in the sample is proportional to the detected hybridization signal. The skilled person will understand that the proportionality need not be strict (e.g., a doubling in transcription rate resulting in a doubling in mRNA transcript and a doubling in hybridization signal). A more relaxed proportionality, for example, where a 10-fold difference in concentration of the target mRNA results in a 5 to 15-fold difference in hybridization intensity may be acceptable. Where more precise quantification is required appropriate controls can be run to correct for variations introduced in sample preparation and hybridization.

Hybridization

In the methods provided herein, the transcript-specific elements from a diseased tissue sample are hybridized to the array under conditions selected to provide a suitable degree of stringency. The skilled person is well aware of techniques for varying hybridization conditions in order to select the most appropriate degree of stringency for a particular sample. For example, using a non-stringent wash buffer (such as 6×SSPE, 0.01% Tween-20) and a stringent buffer (such as 100 mM MES, 0.1M [Na⁺], 0.01% Tween-20) a person or ordinary skill in the art can alter the number of respective washes (typically 0-20), the wash temperature (typically 15-50° C.) and hybridization temperature (typically 15-50° C.) to achieve optimal hybridization. Methods of optimizing hybridization conditions are well known to those of skill in the art (see, e.g., LABORATORY TECHNIQUES IN BIOCHEMISTRY AND MOLECULAR BIOLOGY, Vol. 24: Hybridization With Nucleic Acid Probes, P. Tijssen, ed. Elsevier, N.Y., (1993)).

In one embodiment, hybridization is performed at low stringency to eliminate mismatched hybrid duplexes with successive washes performed at increasingly higher stringency until a desired level of hybridization specificity is obtained. Hybridization specificity may be evaluated by comparison of hybridization to the gene specific elements with hybridization to various controls that can be present.

Labelling and Detection

The transcript-specific elements hybridized to the nucleic acid elements of the array provided herein are preferably detected by detecting one or more labels attached to the sample transcript-specific elements derived from the diseased tissue sample.

The labels may be incorporated before, during or after hybridization by any suitable means of attaching labels to nucleic acids known in the art. Suitable means may include addition of a label directly to the original transcript-specific element of the sample (e.g., mRNA, polyA mRNA, cDNA, etc.) or to an amplification product during or after amplification of the transcript-specific element of the sample, e.g. using labelled primers or labelled nucleotides.

Labels suitable for use in the methods described herein include, but are not limited to, biotin for staining with labelled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

Depending on the choice of label, the skilled person will be able to choose suitable means for detection of the label well known in the art. For a detailed review of methods of labelling nucleic acids and detecting labelled hybridized nucleic acids see LABORATORY TECHNIQUES IN BIOCHEMISTRY AND MOLECULAR BIOLOGY, Vol. 24: Hybridization With Nucleic Acid Probes, P. Tijssen, ed. Elsevier, N.Y., (1993).

Protein Arrays

In another embodiment, protein arrays are designed and constructed. As used herein, the terms “protein” and “polypeptide” are interchangeable. Tissue-specific elements in these arrays may include proteins, peptides, antibodies, peptide-nucleic acids and the like. Antibodies generated to the encoded polypeptide molecules of the diseased transcriptome may be immobilized on the array in discreet locations and conjugated to polypeptides conjugated with detectable labels specific to the antibodies. A protein isolate from a target sample may be contacted with the labelled array and any displacement of the labelled protein from the immobilized antibody will be visible by a loss of the detectable label in that discreet location on the array. Profiles of protein displacement on the array may be correlated with the responsiveness or unresponsiveness of an individual expressing the array profile to a specific therapeutic agent.

Alternatively, the protein arrays may contain encoded polypeptide molecules of the diseased transcriptome. The polypeptide molecules may be affixed in discreet locations on the transcriptome protein array and detected with antibodies isolated from an individual expressing the diseased transcriptome.

Antibodies can be polyclonal, or more preferably, monoclonal. An intact antibody, or a fragment thereof (e.g., Fab or F(ab′)₂) can be used. The term “labelled”, with regard to the probe or antibody, is intended to encompass direct labelling of the probe or antibody by coupling (i.e., physically linking) a detectable substance to the probe or antibody, as well as indirect labelling of the probe or antibody by reactivity with another reagent that is directly labelled. Examples of indirect labelling include detection of a primary antibody using a fluorescently labelled secondary antibody and end-labelling of a DNA probe with biotin such that it can be detected with fluorescently-labelled streptavidin. The term “biological sample” is intended to include tissues, cells and biological fluids isolated from a subject, as well as tissues, cells and fluids present within a subject. That is, the detection method can be used to detect RNA, protein, or genomic DNA in a biological sample in vitro as well as in vivo. For example, in vitro techniques for detection of RNA include Northern hybridizations and in situ hybridizations. In vitro techniques for detection of protein include enzyme linked immunosorbent assays (ELISAs), Western blots, immunoprecipitations, and immunofluorescence. In vitro techniques for detection of genomic DNA include Southern hybridizations. Furthermore, in vivo techniques for detection of protein include introducing into a subject a labelled antibody. For example, the antibody can be labelled with a radioactive marker whose presence and location in a subject can be detected by standard imaging techniques.

Kits

Kits for detecting the presence of or quantifying transcript-specific elements in a diseased tissue sample are also provided herein. For example, the kit can contain an array of one or more transcriptomes from one or more diseased tissues. The molecules on the array may be polynucleotide, polypeptide or antibody molecules as described herein. The kit optionally also include a detectable label or a labelled compound or agent capable of detecting expression of a gene product in a biological sample and the necessary reagents for labelling the sample and affecting hybridization to complementary sequences on the array. The kit optionally also include means for determining the amount of transcript in the sample, such as a colorimetric chart or device.

More than one array may be included in the kit wherein each array corresponds to a tissue afflicted with different diseases and wherein each array contains a plurality of transcriptomes corresponding to a tissue afflicted with a disease. The compound or agent can be packaged in a suitable container. The kit can further include instructions for using the kit to detect protein or nucleic acid.

Methods of Use for Predictive Medicine

Methods of using the arrays described above in the field of predictive medicine are provided. This field includes diagnostic assays, prognostic assays, predictive assays, pharmacogenomics, and the monitoring of clinical trials for different diseases.

The term “disease” or “disease state” includes all diseases which result or could potentially cause a change of the small molecule profile of a cell, cellular compartment, or organelle in an organism afflicted with the disease. Such diseases may be grouped into three main categories: neoplastic disease, inflammatory disease, and degenerative disease.

Examples of diseases include, but are not limited to, metabolic diseases (e.g., obesity, cachexia, diabetes, anorexia, etc.), cardiovascular diseases (e.g., atherosclerosis, ischemia/reperfusion, hypertension, myocardial infarction, restenosis, cardiomyopathies, arterial inflammation, etc.), immunological disorders (e.g., chronic inflammatory diseases and disorders, such as Crohn's disease, inflammatory bowel disease, reactive arthritis, rheumatoid arthritis, osteoarthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy, etc.), myopathies (e.g. polymyositis, muscular dystrophy, central core disease, centronuclear (myotubular) myopathy, myotonia congenita, nemaline myopathy, paramyotonia congenita, periodic paralysis, mitochondrial myopathies, etc.), nervous system disorders (e.g., neuropathies, Alzheimer's disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, peripheral nervous system diseases and mental disorders such as depression and schizophrenia, etc.), oncological disorders (e.g., leukemia, brain cancer, prostate cancer, liver cancer, ovarian cancer, stomach cancer, colorectal cancer, throat cancer, breast cancer, skin cancer, melanoma, lung cancer, sarcoma, cervical cancer, testicular cancer, bladder cancer, endocrine cancer, endometrial cancer, esophageal cancer, glioma, lymphoma, neuroblastoma, osteosarcoma, pancreatic cancer, pituitary cancer, renal cancer, and the like) and ophthalmic diseases (e.g. retinitis pigmentosum and macular degeneration). The term also includes disorders, which result from oxidative stress, inherited cancer syndromes, and metabolic diseases known and unknown.

In general, the methods of use for predictive medicine are performed as follows: transcript-specific elements from a diseased target cell or tissue or a cell or tissue suspected of a pathological condition are combined with the array described herein and are incubated for a sufficient amount of time under conditions that allow hybridization of the transcript-specific elements to the nucleic acid molecules of the array, and hybridization is detected; detection of hybridization indicates the presence of diseased tissue in the sample or a pattern of transcript expression is analyzed and compared with a reference pattern of transcript-specific element expression from a reference sample to provide information about the sample concerning diagnosis, prognosis, drug screening, resistance, choice of therapy, and the like, as described in more detail below.

Diagnostic Assays

Diagnostic assays utilizing the arrays described herein are provided for determining protein and/or nucleic acid expression and activity in a biological sample (e.g., blood, serum, cells, tissue), to determine whether an individual is afflicted with a disease or disorder or is presymptomatic and at risk of developing a disease or disorder associated with aberrant protein, nucleic acid expression or activity. Early diagnosis will facilitate treatment and enhance the success of therapy and may allow a physician to prophylactically treat an individual even prior to onset of symptoms of the disease or disorder.

The arrays described herein may also be used to identify nucleic acid molecules that are differentially expressed in pathological conditions, such as pathological conditions of a colorectal tissue, lung tissue, breast tissue, liver tissue, or brain tissue.

An exemplary method for detecting the presence or absence of an RNA transcript or gene product in a biological sample involves obtaining a biological sample, which contains nucleic acid elements, from a test subject and contacting the biological sample with a compound or an agent capable of detecting protein or nucleic acid such that the presence of a transcript that hybridizes to an array described herein is detected in the biological sample. An agent for detecting RNA or genomic DNA is preferably a labelled nucleic acid probe capable of hybridizing to RNA or genomic DNA from the sample. The nucleic acid probe can be, for example, a full-length nucleic acid or a portion thereof, such as an oligonucleotide of at least 11, 15, 30, 50, 100, 250, 500, 1,000 or greater nucleotides in length and sufficient to specifically hybridize under stringent conditions to RNA or genomic DNA.

The biological sample is combined with the array to detect transcript-specific elements in the biological sample. In one embodiment, the biological sample contains protein molecules from the test subject. Alternatively, the biological sample contains nucleic acid elements from the test subject such as RNA molecules or genomic DNA molecules. A preferred biological sample is a biological fluid (e.g., serum), cell sample, or tissue biopsy sample isolated by conventional means, such as needle biopsy, from a subject.

The arrays may also be used to identify mutations in a gene that cause production of transcripts present in the transcriptome of diseased tissue. Thus, the invention provides a method for identifying a disease or disorder associated with aberrant RNA expression or activity in which a test sample is obtained from a subject and protein or nucleic acid (e.g., RNA, genomic DNA) is detected, wherein the presence of protein or nucleic acid is diagnostic for a subject having or at risk of developing a disease or disorder associated with aberrant gene expression or activity.

The diagnostic assay provides a method of identifying one or more transcript-specific elements in the sample associated with a predisposition to a pathological condition (such as an early stage cancerous condition that is presymptomatic and undetectable by any other means), or the actual presence of a pathological condition. If the sample hybridization pattern, or pattern of expression, is compared with a reference pattern of transcript-specific element expression from a non-diseased reference sample, a difference in expression between corresponding transcript-specific elements of the target cell and the reference sample is indicative of association with the pathological condition. Likewise, if a pattern of expression is compared with a reference pattern of transcript-specific element expression from a diseased reference sample from a particular pathological condition, the presence of a hybridization pattern or pattern of expression substantially corresponding to that of the reference pattern indicates the presence of the pathological condition or predisposition to the pathological condition in the sample tissue or cell.

The pre-determined reference pattern may compose a pattern of expression across the whole array or of a subset of nucleic acid molecules, for example, a subset determined to have particular relevance to a particular pathological condition. Such novel subsets of nucleic acid molecules may be used in the construction of arrays of nucleic acid elements of relevance to particular pathological conditions. Such novel arrays form a further aspect of the present invention.

Differences in expression may be qualitative or quantitative. For example, the difference may be up-regulation of expression or down-regulation of expression of one or more transcript-specific elements of the target cell of the sample compared to its expression in the reference sample. The measured difference in expression may be an increase in expression of one or more transcripts compared to the expression of the corresponding transcript(s) in the non-diseased reference sample (or control), a decrease in expression of one or more transcripts compared to the expression of the corresponding transcript(s) in the non-diseased reference sample (or control), or an increase in one or more transcripts and a decrease in expression of one or more other transcripts compared to the expression of the corresponding transcript(s) in the non-diseased reference sample (or control). Thus the pattern of modulated expression may be indicative of a particular cell or tissue function.

In a preferred embodiment, the RNA species or gene associated with a pathological condition hybridizes to a nucleotide sequence complementary to one or more sequences from Gene List A-JJ. The pathological condition may be any disease condition. For example, the pathological condition may be cancer. The arrays described herein may be used to distinguish between types of cancer (e.g. breast, colorectal, lung, etc.) as well as subtypes of cancer associated with a given tissue.

In one embodiment, expression of a transcript-specific element is considered to be up-regulated or down-regulated in the target cell if the expression is more than 0.1 fold, 0.5 fold, 1 fold, 1.5 fold, two-fold, five-fold, ten-fold or greater different from that of the corresponding element of the reference sample. Of course, in assessing such quantitative differences, correction factors may be made to measured expression levels, for example based on measured expression of a reference nucleic acid element, which is known to be expressed in both target cell and the reference sample. Any suitable non-diseased reference sample (or control) may be used. For example, the reference sample may be a cell from the same tissue and/or organism and/or subject as the target cell or may contain an average for expression values of such genetic elements in a number of such cells in the absence of the relevant pathology.

As described herein, the arrays described herein enable assessment of expression of a very large proportion of the transcriptome for a particular diseased tissue and thus may be used in the evaluation of patterns of differential expression of a large number of genetic elements associated with a particular pathological condition.

Having determined an association between a transcript or gene and a pathological condition, the presence, copy number or expression level of such a transcript may be used in methods of diagnosis of the presence or of a predisposition to such a condition. Such uses represent further independent aspects of the methods described herein.

Prognostic Assays

Prognostic assays are also provided herein for determining whether an individual who has been diagnosed with a disease or disorder associated with aberrant protein, nucleic acid expression or activity will recover or relapse in the absence of, or after, preliminary medical intervention such as surgery.

The prognostic assays described herein can be used to determine a positive or negative overall survival absent any therapy or after preliminary medical intervention to determine if a predictive assay should be performed to identify the most effect further treatment or treatments. For example, the assays can be used to determine whether a patient should receive surgery only or may be effectively treated with a pharmaceutical agent, biological agent, or therapeutic agent combination cocktail prior to, or following surgery. These assays are particularly useful for individuals with a poor prognosis and who would not recover from a disease or disorder absent treatment and medical intervention. In one such embodiment, hybridization of a transcript-specific element with an disease transcriptome array indicates the likelihood of relapse following surgery or chemotherapy or progression of disease in the absence of surgery or chemotherapy.

In preferred prognostic assays, the array is used to correlate hybridization patterns from the sample with patterns from known diseased tissue that responded adversely or favorably to a particular therapy and did or did not experience a relapse of the disease, such as a relapse of cancer following remission.

Predictive Assays

Predictive assays are also provided for the selection of appropriate therapeutic or prophylactic agents specifically for treating the type of disease or disorder affecting the individual. Therapeutic agents include, but are not limited to, small molecule compounds, agonists, antagonists, proteins (including peptides and antibodies or antibody fragments), peptidomimetics, nucleic acids, gene therapy vectors, radiotherapy, chemotherapy, as well as other therapeutic agent candidates.

The information obtained may then be used to determine the response of a disease-associated tissue to a medical treatment method. These methods include determining the patient response to a particular therapy after tumor resection, after extramural recurrence of a tumor, and a tumor response to radiotherapy, post-operative radiotherapy, or chemotherapy.

As well as enabling the screening of candidate agents for modulatory activity, the arrays described herein may be used as tools in the determination of the mode of action of an agent, for example, a therapeutic agent.

Pharmacogenomics Assays

The arrays described herein are also useful in assays for determining protein, nucleic acid expression or activity resulting from an individual's genotype to determine the ability of the individual to respond to a particular agent and thereby select appropriate therapeutic or prophylactic agents (e.g., drugs) specifically for that individual (referred to herein as “pharmacogenomics”).

In this capacity, the arrays described herein may be used in prognostic or predictive assays to identify a patient's responsiveness or resistance to a particular medical treatment based on genetic profiles. In this assay, historical data of patient responses to medical treatment are correlated with hybridization patterns for transcript-specific elements from diseased tissue samples from those patients. This information may then be used to determine the response of future patients to the same medical treatment. These methods include determining the patient prognosis after tumor resection, after extramural recurrence of a tumor, and a tumor response to radiotherapy, post-operative radiotherapy, or chemotherapy.

Exemplary therapeutic agent treatments to be assayed using the transcriptome arrays provided herein include, but are not limited to, an arthritis medication, a chemotherapy drug, a therapeutic antibody, a therapeutic protein or peptides, a therapeutic nucleotide, an antipsychotic drug, an antidepressant drug, an anti-asthmatic drug, an anti-viral drug, and anti-bacterial drug, an anti-hypertensive drug, a cholesterol-lowering drug or an antifungal drug. The arrays may also be used to identify disease progression, aggressiveness of the disease, and identification of the staging of a tumor recurrence.

The arrays provided herein may also be used to determine the degree of adverse response of an individual to a particular therapeutic agent in order to accurately titrate the dosage at the time of treatment and to provide fewer adverse drug reactions. Different polymorphisms may confer increased or decreased metabolism of a particular therapeutic agent. A standard dose may bring about more adverse effects than usual if normal degradative enzymatic activity is reduced by polymorphism. Genetic polymorphisms in drug metabolizing enzymes, transporters, receptors, and other drug targets are linked to interindividual differences in the efficacy and toxicity of many medications. For example, polymorphism in thiopurine methyltransferase (TPMT) results in altered degradation of the commonly prescribed agent 6-mercaptopurine (McLeod and Yu, 2003, Cancer Invest. 21(4):630-40). This genetic variant has significant clinical implications because patients with functionally relevant homozygous mutations in the TPMT gene experience extreme or fatal toxicity after administration of normal doses of 6-MP. In this embodiment, the pattern of expression of a sample is compared with a reference pattern of transcript expression from a reference sample where the presence of a pattern of expression substantially corresponding to that of one or more of the pre-determined reference patterns indicates the chance that the individual may experience an adverse reaction to the treatment.

In a preferred embodiment, a control sample containing cells or tissue of the target cell or tissue that have not been contacted with the therapeutic agent are also combined with the array for comparative purposes.

The arrays described herein are also useful in the monitoring of clinical trials for new or existing therapies. In particular, the arrays are useful for preselecting patients in a patient population having a pathological condition, or prescreening a patient having a pathological condition, to which an experimental therapeutic agent or other therapeutic agent undergoing clinical trials will be administered to treat the pathological condition, so that the patient will be optimally responsive to the drug.

Drug Discovery and Research Assays

The arrays provided herein may be used in drug discovery and research methods. For example, the arrays may be used to determine responses of one or more transcripts/genes of the transcriptome to experimental therapeutic agents, newly synthesized compounds and other agents of interest. The agents may be known to have therapeutic use or may be newly created candidate therapeutic agents.

Therefore, the arrays described herein are useful for screening one or a large number of candidate agents for the ability to modulate target cell or tissue function. In accordance with the method, a hybridization pattern for a sample treated with the therapeutic agent candidate on one or more of the arrays described herein is compared with a hybridization pattern for an untreated control sample. A difference between hybridized transcript-specific elements of the treated sample and the control sample is indicative of the ability of the candidate agent to modulate the target cell or tissue function.

The compositions and methods provided herein will be described in greater detail by way of specific examples. The following examples are offered for illustrative purposes, and are intended neither to limit nor define the invention in any manner.

### EXAMPLES

### Example 1

### Preliminary Listing of Colorectal Cancer Transcriptome Sequences

The following methods were employed to derive the preliminary colorectal cancer transcriptome array sequences disclosed in European patent applications EP 04105479.2, EP 04105482.6, EP 04105483.4, EP 04105484.2, EP 04105507.0, and EP 04105485.9 and U.S. provisional patent application 60/662,276 and 60/700,293.

Materials and Methods

Filtering of Public Data

All the public expressed sequence tags (ESTs) from all the downloaded libraries were retrieved in FASTA format and all 921 libraries were concatenated into a single sequence file containing 272,686 single ESTs. These ESTs were then filtered using a specific combination of filters within Paracel Filtering Package (PFP) (available at the website www.paracel.com) to ensure that undesired sequence elements did not enter the assembly process. Settings were selected to mask low-complexity regions, vector sequences and repeat sequences. Sequences containing contaminating E. coli sequence, mitochondrial DNA or ribosomal RNA were filtered. Subsequent to these filtration steps, low quality end-regions masked in previous stages and any sequences which consisted primarily of low complexity repeats were removed using the “trimjunk” algorithm (Paracel Filtering Package). Finally, any sequences consisting of fewer than 100 good bases were filtered out.

Filtering of Data

The filtering of the ESTs was carried out on the “Phred” output files rather than the raw FASTA sequence files. The “Phred” files contain quality information about the sequence, i.e. how statistically significant the call for each base was. This allowed the use of an additional filtering algorithm known as “qualclean”. Qualclean excises low quality sequence from the start and end of the sequence files. The other filtering algorithms used were identical to those listed for the public data.

Clustering of Data

The assembly of both the public and the in-house data was carried out using the Paracel software “Paracel Transcript Assembler (PTA)” (see the website www.paracel.com), using a clustering threshold of 50. Those sequences that assembled together (contigs) were BLASTed against the Genbank NT database for annotation purposes and to identify the orientation of the sequence. In the cases where the contigs were identified as being in the reverse orientation compared to that listed in Genbank, the sequence was reverse complimented and both orientations were included in the final data set.

Results

Reassembly of Colorectal Derived Sequences from Public Databases

In order to identify sequences that may be expressed in colorectal tissue, Cancer Genome Anatomy Project (CGAP) gateway at the United States National Institutes of Health website (see the website cgap.nci.nih.gov) was examined for sequence information that had been derived from colorectal tissue, colorectal tumor tissue, or colorectal derived cell lines. A total list of 921 EST libraries was identified using the CGAP. The libraries themselves were then retrieved from the UniGene database. The information was collated in a single database to generate a total of 272,686 individual sequences. The individual sequences were subsequently assembled using the Paracel transcript assembly tool to generate a total of 18,721 contigs and 41,023 singlets. The 18,721 contigs were then compared to the contigs generated from the sequencing project set forth below. This comparison revealed some limited redundancy giving a final number of 16,350 publicly derived contigs.

Identification of Novel Colorectal Expressed Sequences

In order to identify additional transcripts that may be expressed in colorectal tissue, whether normal or malignant, a cDNA library was generated from a pool of RNA that had been derived from over 80 normal and malignant colorectal tumor tissues. This RNA was reverse transcribed and directionally cloned into a cloning vector. The library was subsequently transformed into bacteria and plated to generate individual clones. A total of 50,000 clones were selected and sequenced to determine their identity. The 50,000 clones were subsequently assembled to generate a total of 10,396 unique sequences that were assembled to give 4,129 contigs and 6,267 singlets. The sequence information derived from the 4,129 contigs and the 6,267 singlets was then BLASTed against publicly available databases including Genbank to identify totally novel sequences, and against a database generated from all publicly available colon tissue libraries to identify sequences that had not previously been reported to be expressed in colorectal cancer. From this analysis a total of 2,773 novel sequences were identified that had not previously been reported in Genbank as annotated genes or ESTs.

### Example 2

### Further Identification of Colorectal Cancer Sequences

Additional colorectal sequence information was derived by the identification of other transcripts expressed in colorectal tissue through detection on a microarray containing publicly available information. These sequences compliment the preliminary transcriptome array sequence information to provide a more complete array representing the transcriptome for colorectal cancer.

Method

RNA from 40 colorectal tissues (27 tumor and 13 normal) was labeled and hybridized onto the microarray containing publicly available information. From these arrays a list of transcripts was derived for those targets which were called present and above background in at least one of the arrays (i.e. identifying transcripts expressed in at least one of the colorectal samples).

Initial work using the GI or accession numbers associated with the probe sets on the chip showed some discrepancies between the target sequence and the full sequence of the annotated target. As a result of this, it was decided to use the actual sequence of the targets to interrogate the public sequence databases in order to retrieve those sequences from the public databases which best represent the targets which have been empirically determined by the array experiments to be expressed in colorectal tissue.

These sequences were then extracted from the full sequences and these were BLASTed against the provisional patent sequence list (i.e. those transcripts identified from the in-house sequencing and public database mining). From this a list of 21,909 transcripts was derived not represented in the sequence list of U.S. provisional patent application 60/662,276.

This entire list of sequences was BLASTed against the public EST database (dbEST) with a high stringency applied (90% target coverage). Those sequences that hit against dbEST were then retrieved from the public databases. A collection of 16,377 sequences were successfully retrieved by this method.

The remaining 6,635 sequences were BLASTed against the RefSeq database. A collection of 1,663 of the targets produced a solid hit against RefSeq. Once more, these sequences were retrieved from the public database.

For the remaining 4,972 targets, the GI numbers were extracted, and these were used to retrieve the associated sequences from the public databases.

These three lists of sequences were concatenated together into a single file and reviewed with in-house duplicate sequence detection software. This produced a final list of 22,376 sequences with no duplication.

### Example 3

### Antisense Sequences from the Colorectal Cancer Transcriptome

With the increasing interest in the scientific community in the role of endogenous antisense RNA transcripts, the colorectal cancer database was examined for the presence of antisense transcripts.

Method

Subsequent to assembly, both the in-house and public data contigs were BLASTed against the Genbank NT database for annotation purposes and to identify the orientation of the sequence. In the cases where the contigs were identified as being in the reverse orientation compared to that listed in Genbank, the sequence was reverse complimented and both orientations were included in the final data set. Therefore, antisense and corresponding sense transcripts were combined to form a gene list of 5,672 transcripts (Gene List H).

### Example 4

### Listing of Lung Cancer Transcriptome Sequences

The methods employed to derive the lung cancer transcriptome array sequences described in Gene List I to Gene List O were similar to those used in deriving the colorectal cancer sequences.

These 55,626 lung cancer sequences are the result of an in-house assembly of publicly available lung EST libraries. They are a unique assembly of data previously shown to be implicated in lung cancer. A proportion of these sequences are expressed in lung cancer and have not previously been identified as annotated genes.

Results

Reassembly of Lung-Derived Sequences from Public Databases

In order to identify sequences that may be expressed in lung tissue, the CGAP gateway. was examined for sequence information that had been derived from lung tissue, lung tumor tissue, or lung tumor derived cell lines. A total list of 301 EST libraries was identified using the CGAP gateway. The libraries themselves were then retrieved from the UniGene database. The information was collated in a single database to generate a total of 471,630 individual sequences. The individual sequences were subsequently assembled using the Paracel transcript assembly tool to generate a total of 36,431 contigs and 19,195 singlets.

Identification of Novel Lung Expressed Sequences

In order to identify additional transcripts that may be expressed in lung tissue, whether normal or malignant, a cDNA library was generated from a pool of RNA that had been derived from over 80 normal and malignant lung tumor tissues. This RNA was reverse transcribed and directionally cloned into a cloning vector. The library was subsequently transformed into bacteria and plated to generate individual clones. A total of 4,032 clones were selected and sequenced to determine their identity. The clones were subsequently filtered to generate a total of 3,450 unique sequences that were filtered to give 602 contigs and 1,589 singlets. The sequence information derived from the contigs and the singlets was then BLASTed against publicly available databases including Genbank to identify totally novel sequences, and against a database generated from all publicly available lung tissue libraries to identify sequences that had not previously been reported to be expressed in lung cancer. From this analysis a total of 24 novel sequences were identified that had not previously been reported in Genbank as annotated genes or ESTs.

### Example 5

### Listing of Breast Cancer Transcriptome Sequences

The methods employed to derive the breast cancer transcriptome array sequences described in Gene List P to Gene List V were similar to those used in deriving the colorectal and lung cancer sequences.

These 87,059 breast cancer sequences are the results of an in-house assembly of publicly available breast EST libraries. They are a unique assembly of data previously shown to be implicated in breast cancer. A proportion of these sequences are expressed in breast cancer and have not previously been identified as annotated genes.

Results

Reassembly of Breast-Derived Sequences from Public Databases

In order to identify sequences that may be expressed in breast tissue, the CGAP gateway was examined for sequence information that had been derived from breast tissue, breast tumor tissue, or breast tumor derived cell lines. A total list of 1,130 EST libraries was identified using the CGAP gateway. The libraries themselves were then retrieved from the UniGene database. The information was collated in a single database to generate a total of 288,854 individual sequences. The individual sequences were subsequently assembled using the Paracel transcript assembly tool to generate a total of 17,291 contigs and 24,178 singlets.

Identification of Novel Breast Cancer Expressed Sequences

In order to identify additional transcripts that may be expressed in breast tissue, whether normal or malignant, a cDNA library was generated from a pool of RNA that had been derived from over 120 normal and malignant breast tumor tissues. This RNA was reverse transcribed and directionally cloned into a cloning vector. The library was subsequently transformed into bacteria and plated to generate individual clones. A total of 157,260 clones were selected and sequenced to determine their identity. The clones were subsequently filtered to generate a total of 127,306 unique sequences that were assembled to give 14,489 contigs and 24,308 singlets. The sequence information derived from the contigs and the singlets was then BLASTed against publicly available databases including Genbank to identify totally novel sequences, and against a database generated from all publicly available breast tissue libraries to identify sequences that had not previously been reported to be expressed in breast cancer. From this analysis a total of 3,278 novel sequences were identified that had not previously been reported in Genbank as annotated genes or ESTs.

### Example 6

### Listing of Transcriptome Sequences from Liver Tissue Associated with Hepatitis

The methods employed to derive the transcriptome array sequences for liver tissue associated with hepatitis described in Gene List W to Gene List CC were similar to those used in deriving the colorectal and lung cancer sequences.

These 86,122 diseased liver tissue sequences are the results of an in-house assembly of publicly available liver EST libraries. They are a unique assembly of data previously shown to be implicated in liver tissue associated with hepatitis. A proportion of these sequences are expressed in liver tissue associated with hepatitis and have not previously been identified as annotated genes.

Results

Reassembly of Diseased Liver Sequences from Public Databases

In order to identify sequences that may be expressed in liver tissue associated with hepatitis, public databases were examined for sequence information that had been derived from liver tissue, liver tissue associated with hepatitis, or cell lines derived from liver tissue associated with hepatitis. A total list of 63 EST libraries were identified. The libraries themselves were then retrieved from the UniGene database. The information was collated in a single database to generate a total of 326,079 individual sequences. The individual sequences were subsequently assembled using the Paracel transcript assembly tool to generate a total of 24,744 contigs and 37,503 singlets. The contigs were then compared to the contigs generated from the sequencing project set forth below giving a final number of 24,744 publicly derived contigs.

Identification of Novel Sequences Expressed in Liver Tissue Associated with Hepatitis

In order to identify additional transcripts that may be expressed in liver tissue associated with hepatitis, a cDNA library was generated from a pool of RNA that had been derived from over 40 normal and diseased liver tissue samples. This RNA was reverse transcribed and directionally cloned into a cloning vector. The library was subsequently transformed into bacteria and plated to generate individual clones. A total of 4,944 clones were selected and sequenced to determine their identity. The sequences were subsequently quality filtered to generate a total of 2,869 sequences that were assembled to give 45 contigs and 2,300 singlets. The sequence information derived from the contigs and the singlets was then BLASTed against publicly available databases, including the NCBI RefSeq collection, to identify totally novel sequences, and against a database generated from all publicly available liver tissue libraries to identify sequences that had not previously been reported to be expressed in liver tissue associated with hepatitis. From this analysis a total of 13 novel sequences were identified that had not previously been reported in Genbank as annotated genes or ESTs.

### Example 7

### Listing of Transcriptome Sequences from Brain Tissue Associated with Neurodegeneration

The methods employed to derive the transcriptome array sequences for brain tissue associated with neurodegeneration described in Gene List DD to Gene List JJ were similar to those used in deriving the colorectal and lung cancer sequences.

These 136,326 diseased brain tissue sequences are the results of an in-house assembly of publicly available brain EST libraries. They are a unique assembly of data previously shown to be implicated in brain tissue associated with neurodegeneration. A proportion of these sequences are expressed in brain tissue associated with neurodegeneration and have not previously been identified as annotated genes.

Results

Reassembly of Diseased Brain Tissue Sequences from Public Databases

In order to identify sequences that may be expressed in brain tissue associated with neurodegeneration, public databases were examined for sequence information that had been derived from brain tissue, brain tissue associated with neurodegeneration, or cell lines derived from brain tissue associated with neurodegeneration. A total list of 674 EST libraries was identified using public databases. The libraries themselves were then retrieved from the UniGene database. The information was collated in a single database to generate a total of 656,559 individual sequences. The individual sequences were subsequently assembled using the Paracel transcript assembly tool to generate a total of 33,275 contigs and 65,022 singlets.

Identification of Novel Sequences Expressed in Brain Tissue Associated with Neurodegeneration

In order to identify additional transcripts that may be expressed in brain tissue associated with neurodegeneration, a cDNA library was generated from a pool of RNA that had been derived from over 20 normal and diseased brain tissue samples. This RNA was reverse transcribed and directionally cloned into a cloning vector. The library was subsequently transformed into bacteria and plated to generate individual clones. A total of 7,200 clones were selected and sequenced to determine their identity. The sequences were subsequently quality filtered to generate a total of 3,115 sequences that were assembled to give 346 contigs and 1,671 singlets. The sequence information derived from the contigs and the singlets was then BLASTed against publicly available databases, including the NCBI RefSeq collection, to identify totally novel sequences, and against a database generated from all publicly available brain tissue libraries to identify sequences that had not previously been reported to be expressed in brain tissue associated with neurodegeneration. From this analysis a total of 5 novel sequences were identified that had not previously been reported in Genbank as annotated genes or ESTs.

### Example 8

### Comparison of Sequences from Colorectal, Prostate and Breast Tumors

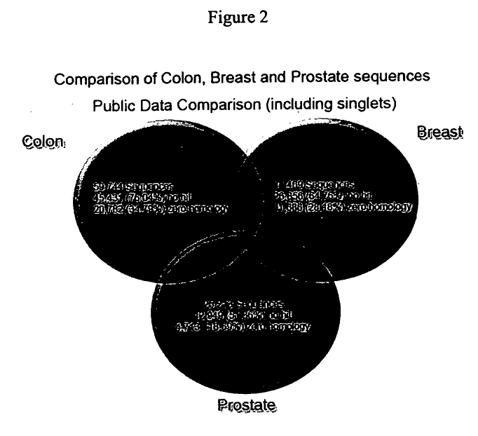

Sequences from colorectal tumor, prostate tumor and breast tumor were compared for commonly expressed sequences. FIG. 2 provides a schematic representation of the BLAST comparisons of all publicly available sequences for colon, prostate and breast tissue. This is a comparison of all sequences post-assembly of the publicly available sequences, obtained as outlined above. The parameters used for BLASTing these sequences were a cut off E-value of 0.1, a percentage identity of 90%. The standard cut off values were derived from manual inspection and visualization of thousands of individual BLAST results. The hits satisfying these criteria can be fairly classified as being “identical” hits while allowing a fair margin for nominal differences that exist between sequences. Hits failing to meet the criteria are different to such a degree that they cannot be considered to be identical for the purposes of array design. Two values are given for each result. The “zero homology” result shows the number of sequences which have no homology whatsoever to the database against which they are BLASTed. The second value is defined as “no hit” and in this case, the query strand has a “percentage coverage” of less than 50%, i.e. the query sequence has less than 50% of its length represented by the target sequences.

The zero homology sequences are a subset of the no-hit sequences. The number of total sequences minus the number of no-hit sequences provides the number of sequences common between the two populations.

All documents referred to in this specification are herein incorporated by reference.

Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

We claim:

1. An array comprising a transcriptome from a diseased tissue.

2. The array of claim 1 wherein the diseased tissue comprises a tissue afflicted with a neoplastic disease, an inflammatory disease or a degenerative disease.

3. The array of claim 1 wherein the diseased tissue comprises a tissue afflicted with colorectal cancer, lung cancer, or breast cancer.

4. The array of claim 1 wherein the transcriptome comprises nucleic acid molecules derived from coding and non-coding transcripts from the diseased tissue.

5. The array of claim 4 wherein the nucleic acid molecules are localized to more than one distinct location on the array.

6. The array of claim 4 wherein the nucleic acid molecules are spotted in one location in the array.

7. The array of claim 1 wherein the transcriptome comprises nucleic acid molecules having sequences complementary to transcripts from diseased colorectal tissue sequences from Gene List B, Gene List C, Gene List D, Gene List E, Gene List F, Gene List G, or Gene List H, or combinations thereof.

8. The array of claim 1 wherein the transcriptome comprises 70% of the nucleic acid molecules having sequences complementary to transcripts from diseased colorectal tissue.

9. The array of claim 1 wherein the transcriptome comprises nucleic acid molecules having sequences complementary to transcripts from diseased lung tissue sequences from Gene List J, Gene List K, Gene List L, Gene List M, Gene List N, or Gene List O, or combinations thereof.

10. The array of claim 1 wherein the transcriptome comprises 70% of nucleic acid molecules having sequences complementary to transcripts from diseased lung tissue.

11. The array of claim 1 wherein the transcriptome comprises nucleic acid molecules having sequences complementary to transcripts from diseased breast tissue sequences from Gene List Q, Gene List R, Gene List S, Gene List T, Gene List U, or Gene List V, or combinations thereof.

12. The array of claim 1 wherein the transcriptome comprises 70% of the nucleic acid molecules having sequences complementary to transcripts from diseased breast tissue.

13. The array of claim 1 wherein the transcriptome comprises nucleic acid molecules having sequences complementary to transcripts from diseased liver tissue sequences from Gene List X, Gene List Y, Gene List Z, Gene List AA, Gene List BB, or Gene List CC, or combinations thereof.

14. The array of claim 1 wherein the transcriptome comprises 70% of the nucleic acid molecules having sequences complementary to transcripts from diseased liver tissue.

15. The array of claim 1 wherein the transcriptome comprises nucleic acid molecules having sequences complementary to transcripts from diseased brain tissue sequences from Gene List EE, Gene List FF, Gene List GG, Gene List HH, Gene List II, or Gene List JJ, or combinations thereof.

16. The array of claim 1 wherein the transcriptome comprises 70% of the nucleic acid molecules having sequences complementary to transcripts from diseased brain tissue.

17. The array of claim 1 wherein the transcriptome comprises nucleic acid molecules having sequences complementary to transcripts from cancer tissues from Gene Lists B, C, D, E, F, G, H, J, K, L, M, N, O, Q, R, S, T, U or V, or combinations thereof.

18. A method for diagnosing a pathological condition in a patient comprising:
a) contacting an array comprising a transcriptome from a diseased tissue with a transcript-specific element from a biological sample from the patient; and
b) detecting hybridization of a transcript-specific element with the array;
wherein detection of hybridization indicates a diagnosis of the pathological condition.

19. A prognostic method for determining whether a patient who has been diagnosed with a disease or disorder will recover or relapse after preliminary medical intervention comprising:
a) contacting an array comprising a transcriptome from a diseased tissue with a transcript-specific element from a biological sample from the patient; and
b) detecting hybridization of a transcript-specific element with the array;
wherein detection of hybridization indicates prognosis for recovery of the pathological condition.

20. A method for determining responsiveness of a patient afflicted with a pathological condition to a therapeutic agent for treatment of the pathological condition comprising:
a) contacting an array comprising a transcriptome from a diseased tissue with a transcript-specific element from a biological sample from the patient; and
b) detecting hybridization of a transcript-specific element with the array;
wherein detection of hybridization indicates responsiveness of the pathological condition of the patient to treatment by the therapeutic agent.
